(19) United States
(12) Patent Application Publication
(10) Pub. No.: US 2006/0183197 A1
(43) Pub. Date: Aug. 17, 2006

(54) Variant growth hormone molecules conjugated with macromolecules compounds

(76) Inventors: Kim Vilbour Andersen, Broenshoej (DK); Joern Drustrup, Farum (DK); Jesper Christiansen, Alleroed (DK)

Correspondence Address: MAXYGEN, INC.;INTELLECTUAL PROPERTY DEPARTMENT, 515 GALVESTON DRIVE, RED WOOD CITY, CA, 94063, US

(21) Appl. No.: 10/250,800

(22) PCT Filed: Jan. 10, 2002
(86) PCT No.: PCT/DK02/00017

§ 371 Date: Dec. 22, 2003

Related U.S. Application Data
(60) Provisional application No. 60/261,411, filed on Jan. 11, 2001.

(51) Int. Cl. C07K 14/61; C07H 21/04; C12N 1/21; C12N 5/06

(52) U.S. Cl. 435/69.4; 530/399; 514/12; 435/320.1; 435/325; 435/252.3; 435/254.2; 536/23.5

(57) ABSTRACT

Conjugates exhibiting growth hormone (GH) activity and comprising at least one non-polypeptide moiety covalently attached to a GH polypeptide, the amino acid sequence of which differs from that of wildtype human GH in at least one introduced and at least one removed amino acid residue comprising an attachment group for said first non-polypeptide moiety. The first non-polypeptide moiety is e.g. a polymer molecule such as PEG or a sugar moiety. The conjugate finds particular use in therapy.

### FIELD OF THE INVENTION

The present invention relates to new polypeptide molecules exhibiting growth hormone (GH) activity, to means and methods for preparing such polypeptide molecules, to pharmaceutical compositions comprising such polypeptides molecules, and the use of such polypeptide molecules in therapy, in particular for the treatment of a variety of disorders caused by growth hormone inadequacy.

### BACKGROUND OF THE INVENTION

Human Growth Hormone (hGH) is a single chain polypeptide hormone comprising 191 amino acid residues and having a Mw of about 22 kDa. hGH is not glycosylated and is synthesized in the somatotropic cells of the anterior pituitary.

Several distinct biological activities have been ascribed to hGH, including effects on linear growth (somatogenesis), tissue growth (skeletal and cell growth), lactation, activation of macrophages, and insulin-like and diabetogenic effects (Chawla et al., Annu. Rev. Med. 34: 519-47, 1983, Edwards et al., Science 239 (4841 Pt1):769-71 (1988), Thorner and Vance, J. Clin. Invest. 82(3): 745-7 (1988). Also, treatment with hGH affects protein, carbohydrate, lipid and mineral metabolism. The biological effects are derived from the interaction between hGH and specific cellular receptors, such as the hGH receptor. For activation of the hGH receptor on cell membranes receptor dimerization is required.

An X-ray structure of a complex between hGH and two copies of the extracellular part of the hGH receptor bound to two different sites on the hGH molecule has been reported by de Vos et al., Science 255, 1992, 306-312. A number of other experimental structures of hGH have been reported in the literature. Details of a hGH structure are given in WO 99/03887, the contents of which are incorporated herein by reference.

A number of recombinant hGH (rhGH) products are on the market, including Humatrope (Eli-Lilly), Nutropin and Protropin (Genentech), Norditropin (Novo-Nordisk), Genotropin (Pharmacia Upjohn), and Saizen or Serostim (Serono). rhGH is used to treat GH deficiency, including treatment of short stature resulting from GH inadequacy and renal failure in children, as well as Turner's syndrome. The protein has a short functional in vivo half-life and must be administered daily by subcutaneous injection for maximum effectiveness (MacGillivray et al., J. Clin, Endocrinol. Metab. 81: 1806-1809). Also, hGH has been approved for treatment of cachexia in AIDS patients and is under study for treating cachexia associated with other diseases. A GH molecule with a longer circulation half-life would decrease the number of necessary administrations and potentially provide more optimal therapeutic hGH levels with concomitant enhanced therapeutic effect.

WO 93/00109 relates to a method for stimulating a mammal's or avian's GH responsive tissues comprising maintaining a continuous, effective plasma GH concentration for a period of 3 or more days. One way of achieving such plasma concentration is stated to be by use of GH coupled to a macromolecular substance such as PEG (polyethylene glycol). The coupling to a macromolecular substance is stated to result in improved half-life.

U.S. Pat. No. 4,179,337 discloses methods of PEGylating enzymes and hormones to obtain physiologically active non-immunogenic, water-soluble polypeptide conjugates. GH is mentioned as one example of a hormone to be PEGylated.

Clark et al., 1996, JBC 271, 36, 21969-21977 discloses PEGylated hGH produced by reaction of hGH with PEG-NHS.

EP 458064 A2 disclose PEGylation of introduced or naturally present cysteine residues in somatotropin. EP 458064 A2 further mentions the incorporation of two cysteine residues in a loop termed the omega loop stated to be located at residues 102-112 in wild type bovine somatotropin, more specifically EP 458064 A2 disclose the substitution of residues numbered 102 and 112 of bovine somatotropin from Ser to Cys and Tyr to Cys, respectively.

WO 9511987 suggest attachment of PEG to the thio group of a cysteine residue being either present in the parent molecule or introduced by site directed mutagenesis. WO 9511987 relates to PEGylation of protease nexin-1, however PEGylation in general of hGH and other proteins is suggested as well.

WO 99/03887 discloses, e.g., growth hormone modified by insertion of additional cysteine residues and attachment of PEG to the introduced cysteine residues.

WO 0042175 relates to a method for making proteins containing free cysteine residues for attachment of PEG. WO 0042175 discloses the following muteins of hGH: T3C, S144C and T148C and the cysteine PEGylation thereof.

WO 9711178 (as well as U.S. Pat. Nos. 5,849,535, 6,004,931, and 6,022,711) relates to the use of GH variants as agonists or antagonists of hGH. WO 9711178 also disclose PEGylation of hGH, including lysine PEGylation and the introduction or replacment of lysine (e.g. K168A and K172R). WO 9711178 also disclose the substitution G120K.

### BRIEF DISCLOSURE OF THE INVENTION

The present invention relates to polypeptide molecules exhibiting GH activity as well as methods for their preparation and their use in medical treatment.

In one aspect the invention relates to a conjugate of a growth hormone polypeptide variant (variant GH) comprising at least one introduced non-cysteine amino acid residue, which residue comprises an attachment group for a macromolecular substance, the residue having been introduced into a position of a parent growth hormone polypeptide (parent GH) that is equivalent to a surface exposed position of wildtype human growth hormone (hGH), the conjugate further comprising at least one macromolecular substance reactive with the non-cysteine amino acid residue.

The invention further relates to a conjugate of a growth hormone polypeptide, wherein the polypeptide comprises at least one amino acid residue with an attachment group for a first macromolecular substance and which amino acid residue is located in a position that is equivalent to a surface exposed position in a helix of hGH, the conjugate further comprising the first macromolecular substance attached to the at least one amino acid residue.

The invention also relates to a conjugate of a variant GH comprising at least one introduced cysteine residue which residue has been introduced in a position equivalent to a position of a parent GH that is equivalent to a position of hGH selected from the group consisting of P2, I4, L6, S7, R8, D11, N12, L15, R16, H18, R19, Q22, F25, D26, Q29, E30, Y35, P37, Y42, L45, L52, E56, S57, P59, S62, N63, R64, E65, E66, Q68, Q69, K70, S71, E74, E88, Q91, F92, R94, S95, L101, Y103, D107, S108, N109, Y111, D112, K115, D116, E119, G120, Q122, T123, G126, R127, R134, Y143, D154, A155, L156, K158, N159, G161, K168, D171, T175, R178, and R183, the conjugate further comprising at least one first cysteine reactive macromolecular substance.

In a still further aspect the invention relates to a conjugate of a growth hormone polypeptide variant (variant GH) comprising at least one removed amino acid residue, preferably a non-cysteine amino acid residue, which residue comprises an attachment group for a (first) macromolecular substance, the residue having been removed from a position of a parent growth hormone polypeptide (parent GH) that is equivalent to a surface exposed position of wildtype human growth hormone (hGH), the conjugate further comprising at least one (first) macromolecular substance attached to an amino acid residue present in said polypeptide, which macromolecular substance is reactive with the removed amino acid residue.

In a further aspect the invention relates to a conjugate of a variant GH comprising at least one introduced in vivo glycosylation site, the in vivo glycosylation site having been introduced in a position of a parent GH that is equivalent to a surface exposed position of hGH, the conjugate further comprising at least one in vivo attached oligosaccharide moiety.

In still further aspects the invention relates to means and methods for preparing a GH molecule of the invention, in particular the conjugate of the invention, including nucleotide sequences, expression vectors and host cells encoding a polypeptide (e.g. a variant GH) of the invention.

The invention also relates to a pharmaceutical composition comprising a GH moleculae of the invention, and optionally a pharmaceutical acceptable acceptable diluent, carrier or adjuvant. In a still further aspect the invention relates to the use of a pharmaceutical composition of the invention and methods for treating a mammal with such composition. In particular the polypeptide, conjugate or composition of the invention may be used to treat diseases or conditions such as those resulting from GH insufficiency or deficiency.

### DETAILED DISCLOSURE OF THE INVENTION

Definitions

In the context of the present application and invention the following definitions apply:

The term “conjugate” (or interchangeably “conjugated polypeptide”) is intended to indicate a heterogeneous (in the sense of composite or chimeric) molecule formed by the covalent attachment of one or more macromolecular substances to a polypeptide, including by in vivo glycosylation. The term “covalent attachment” means that the polypeptide and the macromolecular substance are either directly covalently joined to one another or are indirectly covalently joined to one another through one or more intervening moieties such as a bridge, spacer or linkage moiety. Preferably, the conjugate is soluble at relevant concentrations and conditions, i.e. soluble in physiological fluids such as blood. Examples of conjugated polypeptides of the invention include glycosylated polypeptides and PEGylated polypeptides as well as glycosylated polypeptides having a PEG attached to the sugar moiety.

The term “non-conjugated polypeptide” may be used about the polypeptide part (e.g. the variant GH) of the conjugate.

The term “reactive with” as used herein in the context of the expression “the conjugate comprising a macromolecular substance reactive with the amino acid residue” or similar expressions means that the macromolecular substance is attached to the amino acid residue. Likewise, the term “reactive” in the context of “removal of an amino acid residue comprising an attachment group for a macromolecular substance” means that the removed amino acid residue is one that if it had been present it could have reacted with the macromolecular substance, i.e. the macromolecular substance could have been attached to the attachment group of the amino acid residue had the residue been present in polypeptide part of the conjugate.

The term “macromolecular substance” is intended to indicate a molecule that is capable of conjugating to an attachment group of a polypeptide GH (including a variant GH) in accordance with the invention. The macromolecular substance is typically a non-peptide moiety, i.e. a molecule that is different from a peptide polymer composed of amino acid monomers and linked together by peptide bonds. Preferred examples of macromolecular substances for use herein include polymers, e.g. polyalkylene oxide or oligosaccharide moieties, lipophilic groups, e.g. fatty acids and ceramides.

The term “polymer molecule” is defined as a molecule formed by covalent linkage of two or more monomers and may be used interchangeably with “polymeric group”. Except where the number of macromolecular substances in the conjugate is expressly indicated, every reference to “macromolecular substance” herein is intended as a reference to one or more such substances.

The term “oligosaccharide moiety” is intended to indicate a carbohydrate-containing molecule comprising one or more monosaccharide residues, capable of being attached to the polypeptide (to produce a polypeptide conjugate in the form of a glycosylated polypeptide) by way of in vivo or in vitro glycosylation.

The term “in vivo glycosylation” is intended to mean any attachment of an oligosaccharide moiety occurring in vivo, i.e. during posttranslational processing in a glycosylating cell expressing the polypeptide, e.g. by way of N-linked or O-linked glycosylation. The exact oligosaccharide structure depends, to a large extent, on the glycosylating organism in question.

The term “in vitro glycosylation” is intended to refer to a synthetic glycosylation performed in vitro, normally involving covalently linking an oligosaccharide moiety to an attachment group of a polypeptide, optionally using a cross-linking agent. In vivo and in vitro glycosylation are discussed in detail further below.

An “N-glycosylation site” has the sequence N—X′—S/T/C—X″, wherein X′ is any amino acid residue except proline, X″ any amino acid residue that may or may not be identical to X′ and preferably is different from proline, N asparagine, and S/T/C either serine, threonine or cysteine, preferably serine or threonine, and most preferably threonine. An “O-glycosylation site” is the OH-group of a serine or threonine residue.

The term “attachment group” is intended to indicate a functional group of the polypeptide, in particular of an amino acid residue thereof, or an oligosaccharide moiety, capable of attaching a macromolecular substance such as a polymer molecule, a lipophilic molecule or an organic derivatizing agent. Useful attachment groups and their matching macromolecular substances are apparent from the table below.

| | | Examples of mac- | Conjugation met- | |
|---|---|---|---|---|
| Attachment | | romolecular sub- | hod/-Activated | |
| group | Amino acid | stances | PEG | Reference |
| —NH₂ | N-terminal, | Polymer, e.g. PEG, | mPEG-SPA | Shearwater Inc. |
| | Lys, His, | with arnide or imine | Tresylated mPEG | Delgado et al., |
| | Arg | group | | critical reviews in |
| | | | | Therapeutic Drug |
| | | | | Carrier Systems |
| | | | | 9(3,4):249-304 |
| | | | | (1992) |
| —COOH | C-terminal, | Polymer, e.g. PEG, | mPEG-Hz | Shearwater Inc. |
| | Asp, Glu | with ester or amide | | |
| | | group | | |
| | | Oligosaccharide | In vitro coupling | |
| | | moiety | | |
| —SH | Cys | Polymer, e.g. PEG, | PEG- | Shearwater Inc. |
| | | with disulfide, ma- | vinylsulphone | Delgado et al., |
| | | leimide or vinyl | PEG-maleimide | critical reviews in |
| | | sulfone group | | Therapeutic Drug |
| | | | | Carrier Systems |
| | | Oligosaccharide | | 9(3,4):249-304 |
| | | moiety | In vitro coupling | (1992) |
| —OH | Ser, Thr, | Oligosaccharide | In vivo O-linked | |
| | OH—, Lys | moiety | glycosylation | |
| | | PEG with ester, | | |
| | | ether, carbamate, | | |
| | | carbonate | | |
| —CONH₂ | Asn as part | Oligosaccharide | In vivo N- | |
| | of an N- | moiety | glycosylation | |
| | glycosyla- | | | |
| | tion site | Polymer, e.g. PEG | | |
| Aromatic | Phe, Tyr, | Oligosaccharide | In vitro coupling | |
| residue | Trp | moiety | | |
| —CONH2 | Gln | Oligosaccharide | In vitro coupling | Yan and Wold, |
| | | moiety | | Biochemistry, |
| | | | | 23(16): 3759-65 |
| Aldehyde | Oxidized | Polymer, e.g. PEG, | PEGylation | Andresz et al., |
| Ketone | oligo- | PEG-hydrazide | | 1978, Makromol. |
| | saccharide | | | Chem. 179:301, |
| | | | | WO 92/16555, |
| | | | | WO 00/23114 |
| Guanidino | Arg | Oligosaccharide | In vitro coupling | Lundblad and |
| | | moiety | | Noyes, Chemical |
| | | | | Reagents for Pro- |
| | | | | tein Modification, |
| | | | | CRC Press Inc., |
| | | | | Florida, USA |
| Imidazole | His | Oligosaccharide | In vitro coupling | As for guanidine |
| ring | | moiety | | |

For in vivo N-glycosylation, the term “attachment group” is used in an unconventional way to indicate the amino acid residues constituting an N-glycosylation site. Although the asparagine residue of the N-glycosylation site is where the oligosaccharide moiety is attached during glycosylation, such attachment cannot be achieved unless the other amino acid residues of the N-glycosylation site are present.

Accordingly, when the macromolecular substance is an oligosaccharide moiety and the conjugation is to be achieved by N-glycosylation, the term “amino acid residue comprising an attachment group for the macromolecular substance” as used in connection with alterations of the amino acid sequence of the polypeptide is to be understood as meaning that one or more amino acid residues constituting an N-glycosylation site are to be altered in such a manner that a functional N-glycosylation site is introduced into the amino acid sequence.

For an “O-glycosylation site” the attachment group is the OH-group of a serine or threonine residue, and in that respect the non-polypeptide moiety is an O-linked sugar moiety.

In general, for the conjugate of the invention comprising an introduced amino acid residue with an attachment group for the macromolecular substance, it is preferred that the macromolecular substance is attached to the introduced amino acid residue. More specifically, it it generally understood for the positions specifically indicated herein as attachment sites for the macromolecular substance, that the conjugate of the invention comprises at least the macromolecular substance attached to one of said positions.

The term “mono-PEGylated” is intended to mean that the GH polypeptide has only one polymer comprising a polyethylene glycol (PEG) covalently attached to it. Thus, mono-PEGylated means a polypeptide modified by covalent attachment of a single PEG molecule at a specific site in the polypeptide. Mono-PEGylation means that the conjugate may be homogenous, e.g. the polypeptides are mono-PEGylation in the some position or it may be heterogeneous, e.g. mono-PEGylation of one lysine residue in each polypeptide, for instance, some of the polypeptides may mono-PEGylated in one position whereas other polypeptides are mono-PEGylated in different position.

Amino acid names and atom names (e.g. CA, CB, CD, CG, SG, NZ, N, O, C, etc.) are used as defined by the Protein DataBank (PDB) (www.pdb.org), which is based on the IU-PAC nomenclature (IUPAC Nomenclature and Symbolism for Amino Acids and Peptides (residue names, atom names, etc.), Eur. J. Biochem. 138, 9-37 (1984) together with their corrections in Eur. J. Biochem., 152, 1 (1985). The term “amino acid residue” is intended to indicate an amino acid residue contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W) and tyrosine (Tyr or Y) residues. The terminology used for identifying amino acid positions/substitutions is illustrated as follows: T3 in a given amino acid sequence indicates position number 3 occupied by a threonine residue. T3C indicates that the threonine residue of position 3 has been substituted by a cysteine residue. The numbering of amino acid residues made herein is made relative to the amino acid sequence shown in SEQ ID NO: 2. Multiple substitutions are indicated with a “+”, e.g. T3N+P5S/T means an amino acid sequence which comprises substitution of the Thr residue in position 3 by an Arg residue and substitution of the Pro residue in position 5 by a serine or a threonine residue.

The term “nucleotide sequence” is intended to indicate a consecutive stretch of two or more nucleotide molecules. The nucleotide sequence may be of genomic, cDNA, RNA, semi-synthetic or synthetic origin, or any combination thereof.

The term “polymerase chain reaction” or “PCR” generally refers to a method for amplification of a desired nucleotide sequence in vitro as described, for example, in U.S. Pat. No. 4,683,195. In general, the PCR method involves repeated cycles of primer extension synthesis, using oligonucleotide primers capable of hybridising preferentially to a template nucleic acid.“Cell”, “host cell”, “cell line” and “cell culture” are used interchangeably herein and all such terms should be understood to include progeny resulting from growth or culturing of a cell.

“Transformation” and “transfection” are used interchangeably to refer to the process of introducing DNA into a cell.“Operably linked” refers to the covalent joining of two or more nucleotide sequences, by means of enzymatic ligation or otherwise, in a configuration relative to one another such that the normal function of the sequences can be performed. For example, the nucleotide sequence encoding a presequence or secretory leader is operably linked to a nucleotide sequence for a polypeptide if it is expressed as a preprotein that participates in the secretion of the polypeptide; a promoter or enhancer is operably linked to a coding sequence if it affects the transcription of the sequence; a ribosome binding site is operably linked to a coding sequence if it is positioned so as to facilitate translation.

The term “introduce” is primarily intended to mean substitution of an existing amino acid residue, but may also mean insertion of an additional amino acid residue. Thus, the term “insertion” in this context means in particular that an amino acid residue is introduced between two amino acid residues compared to that of the parent protein, e.g. compared to the equivalent position(s) of hGH.

The term “remove” is primarily intended to mean substitution of the amino acid residue to be removed by another amino acid residue, but may also mean deletion (without substitution) of the amino acid residue to be removed. The term “missing” or “removed” in the context of e.g. the expression “a conjugate of a GH polypeptide variant comprising at least one removed amino acid residue” means the variant GH has at least one removed amino acid residue (such as a non-cysteine residue or a cysteine) compared to that of a the parent GH, e.g. compared to hGH as given by SEQ ID NO:2. In other terms this means that at least one amino acid residue present in an equivalent position of the parent GH is absent from the variant GH, e.g. by deletion or by substitution with a different type of amino acid residue.

It is understood that the terms “introduced” (including “insertion” or “substitution”) and “removal” (including “deletion” or substitution”) as used herein in relation to a variant GH and parent GH are meant to indicate the difference in the amino acid sequence of the variant GH of the invention as compared to that of a parent GH, in particular as compared to the amino acid sequence of hGH as indicated in SEQ ID NO: 2. Thus, preferably these terms are not intended to indicate any limitation as to how the variants are obtained, i.e. whether they are made by mutation of hGH, from another parent GH molecule or by any other methods.

The expression “which amino acid residue is located in a position that is equivalent to a surface exposed position in a helix of hGH” means that the amino acid residue is located at a position which is both in a helix of hGH and at the same time at a surface exposed position of hGH; in the present context this may also be termed “which amino acid residue is located in a position that is equivalent to a position in a surface exposed helix of hGH” or “which amino acid residue is located in a position that is equivalent to a surface exposed position of hGH and in a helix of hGH”.

The term “functional in vivo half-life” is used in its normal meaning, i.e. the time at which 50% of the biological activity of the molecule is still present in the body/target organ, or the time at which the activity of the polypeptide or conjugate is 50% of the initial value.

The term “serum half-life” is used in its normal meaning, i.e. the time in which 50% of the polypeptide or conjugate molecules circulate in the plasma or bloodstream prior to being cleared. Alternative terms to serum half-life include “plasma half-life”, “circulating half-life”, “serum clearance”, “plasma clearance” and “clearance half-life”. Determination of serum half-life is often more simple than determining the functional in vivo half-life, and the magnitude of serum half-life is usually a good indication of the magnitude of functional in vivo half-life.

Functional in vivo half-life and serum half-life may be determined by any suitable method known in the art as further discussed in the Methods section below. The term “increased” as used about the functional in vivo half-life or serum half-life is used to indicate that the relevant half-life of the molecule is statistically significantly increased relative to that of a reference molecule.

A “reference molecule” is normally recombinant hGH (e.g. produced in E. coli or in CHO cells) with or without an N-terminal methionine residue, or any other presently available commercial hGH product, e.g. any of those listed in the Background section above, as determined under comparable conditions.

Clearance mechanisms of relevance for a conjugate of the invention may include one or more of the reticuloendothelial systems (RES), kidney, spleen or liver, receptor-mediated degradation, or specific or non-specific proteolysis. The term “renal clearance” is used in its normal meaning to indicate any clearance taking place by the kidneys, e.g. by glomerular filtration, tubular excretion or tubular elimination. Normally, renal clearance depends on physical characteristics of the conjugate, including molecular weight, size (relative to the cutoff for glomerular filtration), symmetry, shape/rigidity and charge. An apparent molecular weight, of about 67 kDa is normally considered to be a cut-off-value for renal clearance. Renal clearance may be measured by any suitable assay, e.g. an established in vivo assay. For instance, renal clearance is determined by administering a labelled (e.g. radiolabelled or fluorescence labelled) polypeptide conjugate to a patient and measuring the label activity in urine collected from the patient. Reduced renal clearance is determined relative to the reference molecule. Preferably, the conjugate of the invention has reduced renal clearance of at least 50%, preferably by at least 75%, and most preferably with at least 90% as compared to the reference molecule as determined under comparable conditions.

The term “immunogenicity” is intended to indicate the ability of the substance to induce a response from the immune system, in particular the capability of a molecule to give rise to the formation of antibodies in a patient to which the molecule is administered and/or the capability to react with antibodies raised against the molecule. The immune response may be a cell or antibody mediated response (see, e.g., Roitt: Essential Immunology (8^{th} Edition, Blackwell) for further definition of immunogenicity). Immunogenicity may be determined by use of any suitable method known in the art, e.g. in vivo or in vitro.

The term “exhibiting GH activity” is intended to indicate that the polypeptide has at least one of the biological properties of hGH, including but not limited to the ability to bind to a GH receptor, the ability to bind to a prolactin receptor, and the ability to induce dimerization of such receptors. Preferably, the conjugate has in vivo or in vitro activity qualitatively comparable to that of hGH. Furthermore, it is desirable that the specific activity of a conjugate of the invention is at the same level or higher than that of reference molecule. The GH activities may be determined in accordance with methods known in the art, including as described in the Methods section below.

As used herein the term “treatment” also include prevention of diseases and the term “disease” also include “disorder” as the case may be.

The term “parent” is used about the GH polypeptide to be modified in accordance with the present invention. While the parent polypeptide may be of any origin, mammalian GH is preferred. The parent GH may be a wildtype mammalian GH (e.g. of rodent, primate or farm animal origin) or a variant thereof.

A “variant” is a polypeptide exhibiting GH activity, which variant has an amino acid sequence that differs in one or more amino acid residues from that of the parent polypeptide, normally in up to 15 amino acid residues, preferably by at most 10, e.g. by at most 5 amino acid residues from that of SEQ ID NO: 2. Accordingly, the amino acid sequence of the GH polypeptide of the invention may, e.g., differ in 1, 2, 3, 4, 5, 6, 7, 8, 9, or 10 amino acid residues from that of a parent polypeptide. In a preferred embodiment, the polypeptide of the invention has an amino acid sequence which differs by at least one amino acid residues from that of SEQ ID NO: 2.

Preferably, the parent GH is of human origin, in particular being hGH or a variant thereof. The amino acid sequence of mature hGH is shown in SEQ ID NO: 2. One example of a variant hGH is one having the amino acid sequence shown in SEQ ID NO: 2 with an inserted N-terminal methionine.

The term “differs from” or “comprising” as used in connection with specific mutations/positions is intended to allow for additional differences being present apart from the specified amino acid difference. For instance, in addition to the removal and/or introduction of amino acid residues comprising an attachment group for the macromolecular substance, the GH polypeptide of the invention may comprise other substitutions that are not related to introduction and/or removal of such amino acid residues comprising attachment groups for the macromolecular substance.

The sequence numbering as used herein is generally according to the amino acid sequence of mature hGH shown in SEQ ID NO: 2.

The term GH molecule is intended to indicate any molecule with GH activity, typically a GH polypeptide conjugate or a glycosylated GH polypeptide of the invention.

A variant GH is a polypeptide modified in accordance with the invention (by introduction and/or removal of attachment groups for a macromolecular substance).

The term “conservative” as used about an amino acid substitution is intended to have its normal meaning. Examples of conservative substitutions are within the group of basic amino acids (such as arginine, lysine and histidine), acidic amino acids (such as glutamic acid and aspartic acid), polar amino acids (such as glutamine and asparagine), hydrophobic amino acids (such as leucine, isoleucine and valine), aromatic amino acids (such as phenylalanine, tryptophan and tyrosine), and small amino acids (such as glycine, alanine, serine, threonine and methionine). Amino acid substitutions that do not generally alter the specific activity are known in the art and are described, for example, by H. Neurath and R. L. Hill, 1979, In, The Proteins, Academic Press, New York. The most commonly occurring exchanges are Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Ala/Glu, and Asp/Gly as well as these in reverse.

Sequence List:

SEQ ID NO: 1 Disclose the complete amino acid sequence of hGH (DeNoto et al., Nucleic. Acids. Res. 9:3719-3730(1981):

MATGSRTSLL LAFGLLCLPW LQEGSAFPTI PLSRLFDNAM LRAHRLHQLA
FDTYQEFEEA YIPKEQKYSF LQNPQTSLCF SESIPTPSNR EETQQKSNLE
LLRISLLLIQ SWLEPVQFLR SVFANSLVYG ASDSNVYDLL KDLEEGIQTL
MGRLEDGSPR TGQIFKQTYS KFDTNSHNDD ALLKNYGLLY CFRKDMDKVE
TFLRIVQCRS VEGSCGF

SEQ ID NO: 2 Disclose the mature amino acid sequence of hGH:

FPTIPLSRLF DNAMLRAHRL HQLAFDTYQE FEEAYIPKEQ KYSFLQNPQT
SLCFSESIPT PSNREETQQK SNLELLRISL LLIQSWLEPV QFLRSVFANS
LVYGASDSNV YDLLKDLEEG IQTLMGRLED GSPRTGQIFK QTYSKFDTNS
HNDDALLKNY GLLYCFRKDM DKVETFLRIV QCRSVEGSCG F

SEQ ID NO: 3 Disclose the coding sequence of hGH

Conjugate of the Invention:

In one aspect the invention relates to a conjugate of a growth hormone polypeptide variant (variant GH) comprising an amino acid sequence that differs from the amino acid sequence of a parent growth hormone (preferably SEQ ID NO: 2) by at least one introduced or removed non-cysteine amino acid residue, which residue comprises an attachment group for a macromolecular substance, the residue having been introduced into or removed from a position of a parent growth hormone polypeptide (parent GH) that is equivalent to a surface exposed position of wildtype human growth hormone (hGH), the conjugate further comprising at least one macromolecular substance reactive with the non-cysteine amino acid residue.

Removal of a non-cysteine amino acid residue is contemplated for modulating the conjugation sites of the polypeptide of the invention. For instance, it may be desired to remove specific sites available for lysine PEGylation to reduce the amount and/or control the positions of the polypeptide being PEGylated. In this context, the expression “the conjugate further comprising at least one macromolecular substance reactive with the non-cysteine amino acid residue” means that the polypeptide in another position comprise at least one non-cysteine amino acid residue which is reactive, i.e. which is conjugated, with the macromolecular substance.

By removing and/or introducing amino acid residues comprising an attachment group for the macromolecular substance it is possible to specifically adapt the polypeptide so as to make the molecule more susceptible to conjugation to the non-polypeptide moiety of choice, to optimise the conjugation pattern (e.g. to ensure an optimal distribution of non-polypeptide moieties on the surface of the GH polypeptide and thereby, e.g., effectively shield epitopes and other surface parts of the polypeptide without significantly impairing the function thereof). By removal of one or more attachment groups it is possible to avoid conjugation to the non-polypeptide moiety in parts of the polypeptide in which such conjugation is disadvantageous, e.g. to an amino acid residue located at or near a functional site of the polypeptide (since conjugation at such a site may result in inactivation or reduced activity of the resulting conjugate due to impaired receptor recognition). Further, it may be advantageous to remove an attachment group located closely to another attachment group in order to avoid heterogeneous conjugation to such groups.

Accordingly, the invention relates to a conjugate of a growth hormone polypeptide variant (variant GH) comprising at least one introduced non-cysteine amino acid residue, which residue comprises an attachment group for a macromolecular substance, the residue having been introduced into a position of a parent growth hormone polypeptide (parent GH) that is equivalent to a surface exposed position of wildtype human growth hormone (hGH), the conjugate further comprising at least one macromolecular substance reactive with the non-cysteine amino acid residue.

The non-cysteine amino acid residue is any amino acid residue comprising an attachment group for a macromolecular substance, which amino acid residue is different from cysteine. Examples of such are given in the table shown in the definitions section above.

For instance, when the macromolecular substance is a polymer molecule, such as a polyethylene glycol or polyalkylene oxide derived molecule or an in vitro attached oligosaccaride moiety, the non-cysteine amino acid residue may be selected from the group consisting of lysine, aspartic acid, glutamic acid and arginine. When the macromolecular substance is an oligosaccharide moiety the attachment group is, e.g., an in vivo glycosylation site,-preferably an N-glycosylation site.

Whenever an attachment group for a macromolecular substance is to be introduced into or removed from the GH polypeptide in accordance with the present invention, the position of the polypeptide to be modified is conveniently selected as follows:

Preferably, the position is equivalent to a position that is located at the surface of hGH, and more preferably occupied by an amino acid residue having more than 25% of its side chain exposed to the solvent, preferably more than 50% of its side chain exposed to the solvent. The surface exposed position may be determined by analysis of a three-dimensional structure of hGH alone, or of hGH in complex with its two receptor molecules. Surface exposed positions determined from such structures are described in Example 1 below. Amino acid residues having more than 25% or more than 50% of their side chains exposed to the surface are also described.

Furthermore, it is preferred that the amino acid residue comprising an attachment group for a macromolecular substance as described herein, e.g. a non-cysteine amino acid residue or a Cys, is introduced in a position equivalent to a position located outside a receptor-binding site of hGH as defined in Example 1, at least not in receptor binding site 1, and for conjugates of the invention having hGH agonist activity also outside receptor binding site 2. In this context is understood that the introduced amino acid residue reactive with the macromolecular substance, if present, would be attached to the macromolecular substance.

In further embodiments, it is preferred that the amino acid residue comprising an attachment group for a macromolecular substance as described herein, e.g. a non-cysteine amino acid residue, is introduced in a position equivalent to a position located in a helix of hGH. In particular, the amino acid residue may be introduced in a surface exposed position in a helix of hGH, preferably in a surface exposed position of a helix selected from the group consisting of A, B, C and D. Preferably said position is equivalent to a position of hGH that has more than 25% of its side chain exposed at the surface, preferably more than 50% of its side chain exposed at the surface, e.g. in a model structure of hGH alone or in a model structure of hGH complexed to its two receptor molecules; such positions are described in Example 1. In a preferred embodiment, the position in the helix is equivalent to a position located outside a receptor binding site of hGH, cf. Example 1 for examples of specific positions.

An “equivalent position” is intended to indicate a position in the amino acid sequence of a given parent GH, which is homologous (i.e. corresponding in position in either primary or tertiary structure) to a position in the amino acid sequence shown in SEQ ID NO: 2. The “equivalent position” is conveniently determined on the basis of an alignment of members of the GH sequence family, e.g. using the program CLUSTALW version 1.74 using default parameters (Thompson et al., 1994, CLUSTAL W: improving the sensitivity of progressive multiple sequence alignment through sequence weighting, position-specific gap penalties and weight matrix choice, Nucleic Acids Research, 22:4673-4680) or from published alignments. In order to determine an optimal distribution of attachment groups, the distance between amino acid residues located at the surface of the polypeptide is calculated on the basis of a 3D structure of the polypeptide. More specifically, the distance between the CB's of the amino acid residues comprising such attachment groups, or the distance between the functional group (NZ for lysine, CG for aspartic acid, CD for glutamic acid, SG for cysteine) of one and the CB of another amino acid residue comprising an attachment group are determined. In case of glycine, CA is used instead of CB. In the polypeptide GH (e.g. the variant GH) of a conjugate of the invention, any of said distances is preferably more than 8 Å, in particular more than 10Å in order to avoid or reduce heterogeneous conjugation.

In further embodiments, the invention relates to a conjugate of a growth hormone polypeptide, wherein said polypeptide comprises at least one amino acid residue with an attachment group for a (first) macromolecular substance, which amino acid residue is located in a position that is equivalent to a surface exposed position in a helix of hGH, the conjugate further comprising the macromolecular substance attached to the at least one amino acid residue. Preferably the position is not located in a position equivalent to the first three or last three amino acid residues of the helix. In further embodiments, the position is not located in a position equivalent to the first four or last four amino acid residues of the helix. It is understood that this limitation in a preferred embodiment, is intended to cover only the case where the macromolecular substance is to be attached to these positions. Thus, other types of modification of the parent GH not related to the attachment of the macromolecular substance may be present in the variant GH in the first three or last three amino acid residues of the helices corresponding to that of hGH.

The position equivalent to a position located in a helix (preferably selected from the group consisting of A, B, C, and D) of hGH, in particular located at the surface of hGH as described herein, may for example be determined by analysis of a three-dimensional structure of hGH alone, or of hGH in complex with its two receptor molecules (“B” and “C”) or each receptor molecule alone, e.g. as disclosed in Vos et.al. science (1992) 255, 306-312. In one embodiment, the positions of the helices in hGH is as follows in the table below:

| | Amino acid residue number in |
|---|---|
| hGH | SEQ ID NO: 2 |
| A Helix | 9-34 |
| A-B Loop | 35-71 |
| B Helix | 72-92 |
| B-C Loop | 93-105 |
| C Helix | 106-128 |
| C-D Loop | 129-154 |
| D Helix | 155-184 |

In one embodiment, the amino acid residue (i.e. comprising the attachment group for the macromolecular substance) in the conjugate of the invention is located in a position that is equivalent to a position in hGH selected from the group consisting of 12-31, 75-89, 109-125, and 158-181 (SEQ ID NO: 2), e.g. selected from the group consisting of N12, L15, R16, H18, R19, Q22, F25, D26, Q29, E30, E88, N109, Y111, D112, K115, D116, E119, G120, Q122, T123, K158, N159, G161, K168, D171, T175, and R178, preferably selected from the group the group consisting of E30, E88, N109, Y111, D112, K115, Q122, K158, N159, and G161. In specific embodiments, this amino acid residue is a non-cysteine amino acid residue, e.g. selected from the group consisting of a Lys, Asp, Glu, Ser, Thr, Phe, Tyr, Trp, Gln, Arg and His. Also contemplated is embodiments of the invention, wherein this amino acid residue comprising an attachment group for the macromolecular substance has been introduced into the position equivalent to a position in a helix of hGH as described herein, i.e. in particular a surface exposed position in a helix of hGH, e.g the positions as is described above. In other specific embodiments, this introduced amino acid residue is a Cys as also described herein.

In further embodiments, the amino acid residue with the attachment group for the macromolecular substance) and which is in a position that is equivalent to a surface exposed position in a helix of hGH, is not located in helix C, or at least only in a position equivalent to a position loacted outside a receptor binding site of hGH, or at least not in a position equivalent to a receptor binding site, e.g. at least not in the position G120. Thus, in one embodiment, the amino acid residue is located in a helix selected from the group consisting of A, B, or D.

In a particularly preferred embodiment, the introduced amino acid residue with the attachment group for the macromolecular substance is located in a position eqivalent to a surface exposed position of helix B, preferably in a position selected from the group consisting of: E74, E88, Q91 and F92. Particularly preferred is position E88 or Q91, e.g. E88C, E88K, Q91C, or Q91K.

Accordingly, in one embodiment, the introduced amino acid residue with the attachment group for the macromolecular substance is located in a position eqivalent to E74, e.g. E74C. In another embodiment the introduced amino acid residue with the attachment group for the macromolecular substance is located in a position eqivalent E91, e.g. E91C.

In one embodiment, the polypeptide part of the conjugate of the invention does not have a Cys residue in a position that is equivalent to a position in hGH selected from 100 to 111 (SEQ ID NO: 2).

Also, when an amino acid residue comprising the attachment group for a macromolecular substance, e.g. a non-cysteine amino acid residue, is to be introduced into a parent GH by substitution, the amino acid residue to be substituted may be one which can be conservatively substituted with the amino acid residue comprising the attachment group for the macromolecular substance.

As indicated above, in addition to or as an alternative to introducing non-cysteine amino acid residues comprising an attachment group for the macromolecular substance, amino acid residues comprising such attachment group and located at a functional site of the parent GH, e.g. the receptor binding site (e.g. in one or more of the positions K41, K168 and K172), may be removed, preferably by conservative substitution of the amino acid residue comprising such group or by deletion.

Accordingly, the invention also relates to a conjugate of a growth hormone polypeptide variant (variant GH) comprising at least one removed amino acid residue, which residue comprises an attachment group for a (first) macromolecular substance, the residue having been removed from a position of a parent growth hormone polypeptide (parent GH) that is equivalent to a surface exposed position of wildtype human growth hormone (hGH), the conjugate further comprising at least one (first) macromolecular substance attached to an amino acid residue present in said polypeptide, which macromolecular substance is reactive with the removed amino acid residue. In a preferred embodiment, the at least one removed amino acid residue is a non-cysteine amino acid residue.

Accordingly in further embodiment, the variant GH is missing at least one non-cysteine amino acid residue comprising an attachment group for said macromolecular substance as compared to the corresponding parent GH. In other words at least one non-cysteine amino acid residue, e.g. 1, 2, 3, 4 or 5 residues, has been removed from the parent GH. Preferably, the residue(s) comprising an attachment group for said macromolecular substance and which is to be removed is a residue forming part of a functional site, such as a receptor-binding site, of the parent GH.

The removal of one or more the residue(s) comprising an attachment group for the macromolecular substance may be the only modification of attachment groups for the macromolecular substance carried out to prepare the variant GH. Alternatively, the removal of one or more the residue(s) comprising an attachment group for the macromolecular substance may be performed in combination with introduction of one or more residue(s) comprising an attachment group for the macromolecular substance. For instance, introduction and/or removal of attachment groups are designed so as to create a variant GH having attachment groups distributed at the surface of the molecule.

In one embodiment, 1-5, e.g. 1-3, such as only 1, 2, or 3 amino acid residues reactive with the macromolecular substance has been removed from the parent GH.

It is preferred that the removed amino acid residues reactive with the macromolecular substance is located in a surface exposed position equivalent to that of hGH as indicated herein, i.e. e.g. a position equivalent to a position that is located at the surface of hGH, and more preferably occupied by an amino acid residue having more than 25% of its side chain exposed to the solvent, preferably more than 50% of its side chain exposed to the solvent.

In further embodiments, the removed amino acid residues reactive with the macromolecular substance is also located in position equivalent to that of a helix as described herein.

The total number of amino acid residues to be altered in accordance with the present invention, e.g. as described in the subsequent sections herein, (as compared to the amino acid sequence shown in SEQ ID NO: 2) preferably does not exceed 15.

The exact number of amino acid residues and the type of amino acid residues to be introduced depend, i.a., on the desired nature and degree of conjugation (e.g. the identity of the macromolecular substance, how many macromolecular substances it is desirable or possible to conjugate to the polypeptide, where in the polypeptide conjugation should be performed or avoided, etc.).

Preferably, the polypeptide GH (i.e. the variant GH in the present context) of the conjugate of the invention comprises an amino acid sequence which differs in 1-15 amino acid residues from the amino acid sequence shown in SEQ ID NO: 2, such as in 1-8 or 2-8 amino acid residues, e.g. in 1-5 or 2-5 amino acid residues. Thus, preferably the variant GH comprises an amino acid sequence which differs from the amino acid sequence shown in SEQ D NO: 2 in a total of 1, 2, 3, 4, 5, 6, 7, 8, 9, 10, 11, 12, 13, 14 or 15 amino acid residues. Preferably, for the conjugate of the invention at least some of the amino acid residues differing from that of SEQ ID NO: 2 is attached to the macromolecular substance, e.g. a PEG molecule.

The variant GH may comprise at least one additional amino acid change which is not a residue reactive with the macromolecular substance, e.g. 1, 2, 3, or 4 additional amino acid changes compared to hGH, which additional amino acid change(s), e.g. confers antagonist properties, to the GH variant and the corresponding conjugate, e.g. a substitution in the positions equivalent to Gly120 of hGH, e.g. G120R, G120K, G120W, G120Y, G120F, G120E; see for example Fuh et al. (1992) Science, 256, 1677-1680 or WO 9711178.

Also included are a conjugate of the invention comprising a (i.e. one or more) substitution selected from the group consisting of H18D, H18A, H21N, Q22A, F25A, D26A, Q29A, E65A, R167N, K168A, D171S, K172R, E174S, E174A, and I179T, preferably wherein the macromolecular substance of the conjugate is not reactive with the indicated inserted amino acid residue. These substitution may be in addition to the insertion of an amino acid residue comprising an attachment group as described herein, e.g. G120C.

In one embodiment, the conjugate of the invention comprises the substitution G120C as well as a cysteine reactive macromolecular substance, preferably a PEG, attached to said position.

In a preferred embodiment, the GH antagonist of the invention is capable of binding to the hGH receptor(s), at least one of the receptor site 1 and 2 is capable of binding to hGHR (i.e. preferably at least receptor site 1) but incapable of activating the intracellular signalling pathways.

It is understood that the conjugates of the invention exhibits growth hormone (GH) activity, as an agonist or as an antagonist, as also described herein.

In some embodiments, the GH polypeptide (including the variant GH) of the invention or the conjugate of the invention may be a hGH antagonist, for example for use in the treatment of diseases which involves excess production of OH, e.g. cancer or inflammation conditions.

In one embodiment, the conjugation to the macromolecular substance, preferably a PEG group or a sugar moiety attached by in vivo glycosylation, (whether attached to an introduced or not introduced amino acid residue) confers hGH antagonist properties to the conjugate as compared to the unconjugated GH polypeptide of the invention or preferably as compared to unconjugated hGH. In the section “Receptor binding site” in Example 1 is described examples of possible attachment sites to confer hGH agonist properties. Thus, the invention also relates to a conjugate of the invention, in particular a conjugate of a growth hormone polypeptide, comprising at least macromolecular substance attached to a position that is equivalent to a surface exposed position in the receptor binding site 2 (i.e. the low affinity site) of hGH, the conjugate having hGH antagonist activity. For example the conjugate of the invention may comprise a macromolecular substance as described herein in a position equivalent to a position of hGH selected from the group consisting of: F1, P2, I4, P5, R8, L9, D11, N12, A13, L15, R16, H18, R19, Q22, Y103, D116, L117, E119, G120, T123, L124, and R127; preferably selected from the group consisting of F1, P2, I4, P5, R8, L9, D11, N12, A13, L15, R16, R19, Y103, D116, L117, E119, G120, T123, L124, and R127; selected from the group consisting of: F1, P2, I4, P5, R8, D11, N12, L15, R16, R19, Y103, D116, E119, G120, T123, and R127; or more preferably selected from the group consisting of P2, I4, R8, L15, R16, R19, G120, and T123. The amino acid residue comprising the attachment group for the macromolecular substance is preferably introduced compared to hGH. Accordingly, the invention relates to a conjugate of the invention, wherein the amino acid residue (e.g. a Cys) comprising an attachment group for the macromolecular substance has been introduced into a position equivalent to a surface exposed position in the receptor binding site 2 of hGH and wherein the conjugate possesses hGH antagonist activity. It is preferred that the antagonist conjugate of the invention does not comprise a macromolecular substance attached to the positions equivalent to receptor site 1 of hGH.

Preferably, the conjugate of the invention has one or more improved properties as compared to a reference molecule (as defined herein), as determined under comparable conditions, including increased functional in vivo half-life, increased serum half-life and/or reduced renal clearance. Preferably, the half-life is increased by at least a factor of 2 such as a factor of 5, 10 or more.

Furthermore, it is preferred that the conjugate is not more immunogenic than that of a reference molecule such as hGH, as determined under comparable conditions.

Preferably, the conjugate of the invention comprises a sufficient number or type of macromolecular substances to improve one or more of the above mentioned desired properties of the GH polypeptide. Normally a conjugate of the invention comprises 1-10 (first) macromolecular substances, in particular 1-8 or 1-5 of such substances, e.g. a total of 1, 2, 3, 4, 5, 6, 7 or 8 macromolecular substances.

The macromolecular substance is preferably attached to the introduced amino acid residue(s) (e.g. a non-cysteine amino acid residue), but may also be attached to other amino acid residues of the variant GH with which it is reactive, i.e. other amino acid residues being of the same type as the introduced amino acid residue(s).

In one embodiment, the conjugate of the invention is mono-PEGylated.

In further embodiments, the conjugate does not comprise a macromolecular substance, e.g. a PEG, attached to an amino acid residue of said polypeptide located outside a position as indicated herein. Accordingly, in one embodiment, the conjugate comprises macromolecular substance(s) attached only to amino acid residue(s) located in position(s) equivalent to surface exposed position(s) of a helix of hGH, preferably wherein the position(s) are not located in a the first three or last three amino acids of said helix.

Thus, the polypeptide part of the conjugate of the invention may comprise amino acid residue(s), i.e. residue(s) comprising the attachment group for the macromolecular substance, introduced only into position(s) equivalent to one of the helices of hGH, preferably selected from the group consisting of A, B, C, and D, e.g. only to one of A, B, C or D.

In a specific embodiment, the polypeptide conjugate of the invention is one which comprises a single PEG molecule attached to the N-terminal of the polypeptide and no other PEG molecules, in particular a linear or branched PEG molecule with a molecular weight of at least about 20 kDa. The polypeptide according to this embodiment may further comprise one or more oligosaccharide moieties attached to an N-linked or O-linked glycosylation site of the polypeptide or oligosaccharide moieties attached by in vitro glycosylation.

In yet another aspect, the invention relates to a conjugate of hGH (SEQ ID NO: 2) polypeptide comprising a macromolecular substance attached to the N-terminal amino acid residue. In a preferred embodiment, the invention relates to a conjugate of hGH (SEQ ID NO: 2) having a single PEG molecule attached to the N-terminal of the polypeptide and no other PEG molecules, in particular a linear or branched PEG molecule with a molecular weight of at least about 20 kDA.

In another embodiment, the polypeptide conjugate of the invention comprises a PEG molecule attached to each of the lysine residues in the variant GH available for PEGylation, in particular a linear or branched PEG molecule, e.g. with a molecular weight of about 5 kDa.

The conjugate of the invention may further comprise at least one second macromolecular substance which is different from said first macromolecular substance. For instance, the conjugate of the invention may comprise 1-10 second macromolecular substances, in particular 1-8 or 1-5 second substances. For instance, when the first macromolecular substance is a polymer molecule of the PEG type, a second macromolecular substance of interest is an oligosaccharide moiety, in particular an in vivo attached oligosaccharide moiety. The in vivo attached oligosaccharide moiety is attached to an introduced in vivo glycosylation site of the polypeptide.

Typically, the conjugate according to the invention has an apparent molecular weight of at least about 67 kDa, preferably at least about 70 kDa, although a lower molecular weight may also give rise to a reduced renal clearance. Polymer molecules, such as PEG, have been found to be particularly useful for adjusting the molecular weight of the conjugate.

It is contemplated that a conjugate of the present invention offers a number of advantages over the currently available GH products, including longer duration between injections.

Conjugate of the Invention Wherein the Macromolecular Substance is Attached to a Lysine or the N-terminal Amino Acid Residue

In a preferred embodiment the conjugate of the invention is one wherein the macromolecular substance is a molecule that has an epsilon amino group as an attachment group. For instance, the variant GH of a conjugate according to this embodiment comprises at least one introduced lysine residue, the residue having been introduced into a position of a parent GH that is equivalent to a surface exposed position of hGH, the conjugate further comprising at least one macromolecular substance reactive with the lysine residue. For instance, a lysine residue has been introduced into at least one position of the parent GH that is equivalent to a position of hGH selected from the group consisting of amino acid residues having at least 25% of its side chain exposed to the surface, preferably at least 50% of its side chain exposed to the surface, e.g. in a model structure of hGH alone or complexed to its receptor molecules. Such amino acid residues are identified in Example 1. Preferably, the lysine residue is introduced by way of substitution of an amino acid residue located in the relevant position(s), in particular by conservative substitution. Example 1 herein lists specific positions suitable for introduction of a lysine residue as well as specific substitutions.

The variant GH of the conjugate according to this embodiment preferably comprises at least one substitution to lysine as identified in Example 1 hereinafter, examples of which are R64K, R94K, R127K, R134K and R183K (the numbering is according to the mature amino acid sequence of hGH as shown in SEQ ID NO: 2, i.e. at an equivalent position).

The variant GH of the conjugate according to this embodiment typically comprises1-10 introduced lysine residues, in particular 1-5 or 1-3, e.g. 1, 2, 3, 4, or 5 introduced lysine residues.

In a further embodiment the variant GH is missing at least one lysine residue as compared to the corresponding parent GH. In other words at least one lysine residue, e.g. 1, 2, 3, 4 or 5 lysine residues, has been removed from the parent GH. In principle any of the lysine residues of the parent GH, in particular the 9 lysine residues of hGH, can be removed in accordance with this embodiment, preferably by substitution, in particular conservative substitution. In Example 1 the 9 amino acid residues of hGH are identified. Preferably, the lysine residue(s) to be removed is a lysine residue forming part of a functional site, such as a receptor-binding site, of the parent GH. For instance, the variant GH according to this embodiment comprises at least one or at least two substitution(s) equivalent to a substitution of hGH selected from the group consisting of K41R, K168R and K172R. In one embodiment, the conjugate of the invention comprises a substitution of the lysine in all of the three positions equivalent to of K41, 168R and 172R, e.g. K41R, K168R and K172R. Preferably, such GH variants with removed lysine residues comprise introduced lysine residues as well.

The removal of one or more lysine residues may be the only modification of attachment groups for the macromolecular substance carried out to prepare the variant GH. Thereby, a lysine reactive macromolecular substance is attached to a remaining naturally-occurring lysine residue of the GH polypeptide, whereas conjugation to the removed lysine residue located, e.g., at a receptor binding site is avoided. Alternatively, the removal of one or more lysine residues may be performed in combination with introduction of one or more lysine residues, e.g. to create a variant GH deleted of lysine residues located in a functional site, such as a receptor-binding site, and added in one or more lysine residues. For instance, introduction and/or removal of attachment groups are designed so as to create a variant GH having attachment groups distributed at the surface of the molecule in accordance with the general guidelines given in the section above entitled “Conjugate of the invention”.

While the macromolecular substance may be any of those binding to a lysine residue, e.g. the ε-amino group thereof, such as a polymer molecule, a lipophilic group, an organic derivatizing agent, it is preferably any of the polymer molecule mentioned in the section entitled “Conjugation to a polymer molecule”, in particular a branched or linear PEG or polyalkylene oxide. Most preferably, the polymer molecule is PEG and the activated molecule to be used for conjugation is SS-PEG, NPC-PEG, aldehyd-PEG, mPEG-SPA, mPEG-SCM, mPEG-BTC from Shearwater Polymers, Inc, SC-PEG from Enzon, Inc., tresylated mPEG as described in U.S. Pat. No. 5,880,255, or oxycarbonyl-oxy-N-dicarboxyimide-PEG (U.S. Pat. No. 5,122,614).

Normally, for conjugation to a lysine residue the macromolecular substance has a molecular weight of about 5 or 10 kDa. The conjugate according to this embodiment may comprise at least one second macromolecular substance, such as 1-10, 1-8 or 1-5 such substances.

When the first macromolecular substance is a polyalkylene oxide or PEG derived polymer, the second macromolecular substance is preferably an oligosaccharide moiety, in particular an in vivo attached moiety, e.g. attached to an introduced in vivo glycosylation site as described in the section entitled “Conjugate of the invention wherein the macromolecular substance is an oligosaccharide moiety”.

Conjugate of the Invention Having Peptide Moiety Attaching to a Non-Cysteine or Non-Lysine Residue

Based on the present disclosure the skilled person will be aware that amino acid residues comprising other attachment groups may be introduced by substitution into the parent GH, using the same approach as that illustrated above with lysine residues. For instance, one or more amino acid residues comprising an acid group (glutamic acid or aspartic acid), or arginine may be introduced into positions which in the parent GH are equivalent to a position of hGH occupied by a surface exposed amino acid residue in particular positions occupied by an amino acid residue having at least 25% of its side chain exposed to the surface, in particular at least 50% of its side chain exposed to the surface. For this purpose it is preferred that the introduction is by substitution, preferably conservative substitution. Analogously to what has been described above for lysine modified conjugates, the resulting modified polypeptide may be conjugated to at least one first macromolecular substance (capable of attaching to the amino acid residue having been introduced) and may further comprise at least one second macromolecular substance, e.g. an in vivo attached oligosaccharide moiety.

Conjugate of the Invention Wherein the Macromolecular Substance Attaches to a Cysteine Residue

In further aspects, the invention relates to a conjugate of a variant GH comprising at least one introduced cysteine residue, which residue has been introduced in a position of a parent GH that is equivalent to a surface exposed position in a helix of hGH provided the position is not located in the first three or last three amino acid residues of the helix, the conjugate further comprising at least one (first) cysteine reactive macromolecular substance. Preferably this position is equivalent to a position of hGH that has more than 25% of its side chain exposed at the surface, preferably more than 50% of its side chain exposed at the surface, in a model structure of hGH alone or complexed to its two receptor molecules.

In a further aspect, the invention relates to a conjugate of a variant GH comprising at least one cysteine residue (e.g. only 1, 2, 3, 4, 5 or 6 introduced Cys) introduced into a position of a parent GH (preferably hGH or a variant thereof differing by at most 10, or at most 8, at most 5, at most 4, at most 3, at most 2, such as 1 or 2 amino acid residues) equivalent to a position of hGH selected from the group consisting of P2, I4, L6, S7, R8, D11, N12, L15, R16, H18, R19, Q22, F25, D26, Q29, E30, Y35, P37, Y42, L45, L52, E56, S57, P59, S62, N63, R64, E65, E66, Q68, Q69, K70, S71, E74, E88, Q91, F92, R94, S95, L101, Y103, D107, S108, N109, Y111, D112, K115, D116, E119, G120, Q122, T123, G126, R127, R134, Y143, D154, A155, L156, K158, N159, G161, K168, D171, T175, R178, and R183, the conjugate comprising at least one first cysteine reactive macromolecular substance, i.e. in particular a free cysteine residue not forming part of a disulfide bridge. In a preferred embodimet, the cysteine reactive macromolecular substance is attached to at least one of the introduced Cys.

In one embodiment, the conjugate of the variant GH comprises at least one cysteine residue (e.g. only 1, 2, 3, 4, 5 or 6 cysteine residues) introduced into a position of a parent GH equivalent to a position of hGH selected from the group consisting of L6, S7, E30, Y35, P37, L52, S57, P59, E66, Q69, K70, S71, E74, E88, Q91, F92, R94, S95, L101, D107, S108, N109, Y111, D112, K115, Q122, G126, R134, Y143, D154, A155, L156, K158, N159, and G161, the conjugate comprising at least one cysteine reactive macromolecular substance.

In further embodiments, the conjugate of such “cysteine” variant GH in addition to the introduction of a Cys into a position selected from the groups as outline above, further comprise at least one additional cysteine residue (e.g. only 1, 2, 3, 4, 5 or 6 additional cysteine residue(s)) introduced into a position of the parent GH (preferably hGH) equivalent to a position of hGH selected from the group consisting of F1, T3, P5, E33, A34, K38, E39, Q40, S43, Q46, N47, P48, Q49, A98, N99, G104, S106, E129, D130, G131, P133, T135, G136, Q137, K140, Q141, K145, D147, E186, G187, and G190; though preferably selected from the group consisting of F1, T3, P5, E33, K38, E39, S43, Q46, N47, P48, Q49, N99, E129, G131, P133, T135, G136, D147, E186, G187, and G190.

Preferably the polypeptide part of the conjugate of the invention does not have a Cys in a position equivalent to Y111, at least not as the only introduced Cys residue. In one embodiment of the invention, the polypeptide part of the conjugate of the invention has a Cys residue in a position that is equivalent to a position in hGH selected from 100 to 111 (SEQ ID NO: 2), e.g. in a position equivalent to Y111 as well as at least one additional introduced Cys as described herein; e.g. selected from the group consisting of N12, L15, R16, H18, R19, Q22, F25, D26, Q29, E30, E88, N109, D111, K115, D116, E119, G120, Q122, T123, K158, N159, G161, K168, D171, T175, and R178, or preferably selected from the group consisting of E30, E88, N109, D112, K115, Q122, K158, N159, and G161.

In addition to the at least one introduced cysteine residue (e.g. a total of 1, 2, 3, 4, 5 or 6 introduced cysteine residue(s)), the variant GH preferably comprises 4 cysteine residues in positions equivalent to Cys 53, Cys 165, Cys 182 and Cys 189 forming disulfide bridges corresponding to that of hGH, i.e. Cys 53 with Cys 165 and Cys182 with Cys 189.

In one embodiment, the conjugate of the invention comprises the substitution G120C, to which position is attached the macromolecular substance, i.e. a cysteine reactive molecule.

In one embodiment, the GH polypeptide of the conjugate of the invention is as described herein provided that the polypeptide comprise at most one cysteine residue, i.e. only one or none, in the positions equivalent to amino acid residues from number 100 to 111.

In further embodiments, the GH polypeptide of the conjugate of the invention is as described herein, provided that the polypeptide does not at the same time comprise a cysteine residue in both of the positions corresponding to 100 and 111 of of hGH.

While the macromolecular substance of the conjugate according to this aspect of the invention may be any molecule which, when using the given conjugation method has a cysteine as an attachment group (such as an oligosaccharide moiety, a lipophilic group or an organic derivatizing agent), it is preferred that the macromolecular substance is a polymer molecule, e.g. any of the molecules mentioned in the section entitled “Conjugation to a polymer molecule”. Preferably, the polymer molecule is selected from the group consisting of linear or branched polyethylene glycol or polyalkylene oxide. Most preferably, the polymer molecule is PEG, such as VS-PEG.

The conjugation between the polypeptide and the polymer may be achieved in any suitable manner, e.g. as described in the section entitled “Conjugation to a polymer molecule”, e.g. in using a one step method or in the stepwise manner referred to in said section. When the polypeptide comprises only one conjugatable cysteine residue, this is preferably conjugated to a first macromolecular substance with a molecular weight of at least 10 or at least 15 kDa, such as a molecular weight of 12 kDa, 15 kDa or 20 kDa, either directly conjugated or indirectly through a low molecular weight polymer (e.g. as disclosed in WO 99/55377). When the conjugate comprises two or more first macromolecular substances, normally each of these has a molecular weight of 5 or 10 kDa.

Conjugate of the Invention Wherein the Macromolecular Substance is an Oligosaccharide Moiety

In a further aspect the invention relates to a conjugate comprising a glycosylated variant GH, wherein the variant GH comprises at least one in vivo glycosylation site. Preferably, the in vivo glycosylation site is introduced into a position equivalent to a position of hGH occupied by a surface exposed amino acid residue (as identified in Example 1). The introduction of a glycosylation site is illustrated below using an in vivo N-glycosylation site as an example. It will be understood that an O-glycosylation site or an in vitro glycosylation site may be introduced in an analogous manner.

A suitable N-glycosylation site may be introduced by introducing, preferably by substitution, an asparagine residue in a position equivalent to a position of hGH occupied by a surface exposed amino acid residue, in particular an amino acid residue having more than 25% of its side chain exposed at the surface of hGH, and preferably more than 50% of its side chain exposed at the surface, which position does not have a proline residue located in position +1 or +3 therefrom. If the amino acid residue located in position +2 is a serine or threonine, no further amino acid substitution is required. However, if this position is occupied by a different amino acid residue, a serine or threonine residue needs to be introduced.

In Example 1 suitable positions for introduction of additional N-glycosylation sites are disclosed. The variant GH of a conjugate of the invention may contain a single in vivo glycosylation site. However, it may be desirable that the polypeptide comprises more than one in vivo glycosylation site, in particular 1-10, such as 2-5 in vivo glycosylation sites. Thus, the GH polypeptide may comprise one additional glycosylation site, or may comprise two, three, four, five, six, seven or more introduced in vivo glycosylation sites.

As indicated herein, the N-glycosylation site is introduced in such a way that the N-residue of said site is located in said position. Analogously, an O-glycosylation site is introduced so that the S or T residue making up such site is located in said position.

Furthermore, in order to ensure efficient glycosylation it is preferred that the in vivo glycosylation site, in particular the N residue of the N-glycosylation site or the S or T residue of the O-glycosylation site, is not located in the last (i.e. in the C-terminal part of the polypeptide) 10, 15, 20, 25, 30, 40 or preferably not in the last 50 amino acid residues of the GH polypeptide of the invention. Thus, it is preferred that the glycosylation site(s) as described herein is located within a position equivalent to the first 180 N-terminal amino acid residues of hGH (SEQ ID NO: 2), more preferably within the first 170, or the 160, or 150 N-terminal amino acid residues.

Still more preferably, the in vivo glycosylation site is introduced into a position wherein only one mutation is required to create the site (i.e. where any other amino acid residues required for creating a functional glycosylation site is already present in the molecule).

Furthermore, the amino acid sequence of the variant GH having at least one of the above mentioned in vivo glycosylation site modifications may differ from that of the parent polypeptide in that at least one attachment group for a second macromolecular substance may have been introduced, e.g. as described in the section entitled “Conjugate of the invention”, “Conjugate of the invention wherein the macromolecular substance is attached to a lysine residue or the N-terminal amino acid residue”, or “Conjugate of the invention having macromolecular substance attached to a non-cysteine or non-lysine residue”, and “Conjugate of the invention wherein the macromolecular substance attaches to a cysteine residue”.

In vivo glycosylation is effected by expression in a glycosylating eukaryotic expression host. The expression host cell may be selected from fungal (filamentous fungal or yeast), insect or animal cells or from transgenic plant cells. In one embodiment the host cell is a mammalian cell, such as a CHO cell, a BHK or a HEK cell, e.g. HEK 293, an insect cell, such as an SF9 cell, or a yeast cell, e.g. S. cerevisiae or Pichia pastoris, or any of the host cells mentioned hereinafter.

In addition to an oligosaccharide moiety, the conjugate according to the aspect of the invention described in the present section may contain additional macromolecular substances, in particular a polymer molecule conjugated to one or more, optionally introduced attachment groups present in the variant GH part of the conjugate, e.g. to increase the molecular weight of the conjugate to about or above 67 kDa.

Macromolecular Substance of the Conjugate of the Invention

As indicated above, the macromolecular substance of the conjugate of the invention is preferably selected from the group consisting of a polymer molecule, a lipophilic compound, and an organic derivatizing agent. All of these substances may confer desirable properties to the polypeptide GH (.e.g. the variant GH) of the conjugate of the invention, in particular an increased functional in vivo half-life and/or an increased serum half-life.

The polypeptide GH (.e.g. the variant GH) of the invention is normally conjugated to only one type of macromolecular substance (a first macromolecular substance), but it may also be conjugated to two or more different types of macromolecular substances (second macromolecular substances), e.g. to a polymer molecule and an oligosaccharide moiety, to a lipophilic group and an oligosaccharide moiety, to an organic derivatizing agent and an oligosaccharide moiety, to a lipophilic group and a polymer molecule, etc. The conjugation to two or more different macromolecular substances may be done simultaneously or sequentially.

### METHODS FOR PREPARING A CONJUGATE OF THE INVENTION

In the following sections “Conjugation to a lipophilic compound”, “Conjugation to a polymer molecule”, and “Conjugation to an organic derivatizing agent”, conjugation to specific types of macromolecular substances is described.

Conjugation to a Lipophilic Compound

The polypeptide and the lipophilic compound may be conjugated to each other either directly or by use of a linker. The lipophilic compound may be a natural compound such as a saturated or unsaturated fatty acid, a fatty acid diketone, a terpene, a prostaglandin, a vitamin, a carotenoid or steroid, or a synthetic compound such as a carbon acid, an alcohol, an amine or sulphonic acid with one or more alkyl, aryl, alkenyl or other multiple unsaturated compounds. The conjugation between the polypeptide and the lipophilic compound, optionally through a linker, may be done according to methods known in the art, e.g. as described by Bodanszky in Peptide Synthesis, John Wiley, New York, 1976 and in WO 96/12505.

Conjugation to a Polymer Molecule

The polymer molecule to be coupled to the polypeptide may be any suitable polymer molecule, such as a natural or synthetic homo-polymer or hetero-polymer, typically with a molecular weight in the range of 300-100,000 Da, such as 300-20,000 Da, more preferably in the range of 500-10,000 Da, even more preferably in the range of 500-5000 Da. Examples of homo-polymers include a polyol (i.e. poly-OH), a polyamine (i.e. poly-NH₂) and a polycarboxylic acid (i.e. poly-COOH). A hetero-polymer is a polymer which comprises different coupling groups, such as a hydroxyl group and an amine group.

Examples of suitable polymer molecules include polymer molecules selected from the group consisting of polyalkylene oxide (PAO), including polyalkylene glycol (PAG), such as polyethylene glycol (PEG) and polypropylene glycol (PPG), branched PEGs, poly-vinyl alcohol (PVA), poly-carboxylate, poly-(vinylpyrolidone), polyethylene-co-maleic acid anhydride, polystyrene-co-maleic acid anhydride, dextran, including carboxymethyl-dextran, or any other biopolymer suitable for increasing functional in vivo half-life and/or serum half-life. Another example of a polymer molecule is human albumin or another abundant plasma protein. Generally, polyalkylene glycol-derived polymers are biocompatible, non-toxic, non-antigenic, non-immunogenic, have various water solubility properties, and are easily excreted from living organisms. PEG is the preferred polymer molecule, since it has only few reactive groups capable of cross-linking compared to e.g. polysaccharides such as dextran. In particular, monofunctional PEG, e.g. methoxypolyethylene glycol (mPEG), is of interest since its coupling chemistry is relatively simple (only one reactive group is available for conjugating with attachment groups on the polypeptide). Consequently, the risk of cross-linking is eliminated, the resulting polypeptide conjugates are more homogeneous and the reaction of the polymer molecules with the polypeptide is easier to control. To effect covalent attachment of the polymer molecule(s) to the polypeptide, the hydroxyl end groups of the polymer molecule must be provided in activated form, i.e. with reactive functional groups. Suitable activated polymer molecules are commercially available, e.g. from Shearwater Polymers, Inc., Huntsville, Ala., USA. Alternatively, the polymer molecules can be activated by conventional methods known in the art, e.g. as disclosed in WO 90/13540. Specific examples of activated linear or branched polymer molecules for use in the present invention are described in the Shearwater Polymers, Inc. 1997 and 2000 Catalogs (Functionalized Biocompatible Polymers for Research and pharmaceuticals, Polyethylene Glycol and Derivatives, incorporated herein by reference). Specific examples of activated PEG polymers include the following linear PEGs: NHS-PEG (e.g. SPA-PEG, SSPA-PEG, SBA-PEG, SS-PEG, SSA-PEG, SC-PEG, SG-PEG, and SCM-PEG), and NOR-PEG), BTC-PEG, EPOX-PEG, NCO-PEG, NPC-PEG, CDI-PEG, ALD-PEG, TRES-PEG, VS-PEG, IODO-PEG, and MAL-PEG, and branched PEGs such as PEG2-NHS and those disclosed in U.S. Pat. No. 5 5,932,462 and U.S. Pat. No. 5,643,575, both of which are incorporated herein by reference. Furthermore, the following publications, incorporated herein by reference, disclose useful polymer molecules and/or PEGylation chemistries: U.S. Pat. Nos. 5,824,778, 5,476,653, WO 97/32607, EP 229,108, EP 402,378, U.S. Pat. Nos. 4,902,502, 5,281,698, 5,122,614, 5,219,564, WO 92/16555, WO 94/04193, WO 94/14758, WO 94/17039, WO 94/18247, WO 94/28024, WO 95/00162, WO 95/11924, WO95/13090, WO 95/33490, WO 96/00080, WO 97/18832, WO 98/41562, WO 98/48837, WO 99/32134, WO 99/32139, WO 99/32140, WO 96/40791, WO 98/32466, WO 95/06058, EP 439 508, WO 97/03106, WO 96/21469, WO 95/13312, EP 921 131, U.S. Pat. No. 5,736,625, WO 98/05363, EP 809 996, U.S. Pat. No. 5,629,384, WO 96/41813, WO 96/07670, U.S. Pat. Nos. 5,473,034, 5,516,673, EP 605 963, U.S. Pat. No. 5,382,657, EP 510 356, EP 400 472, EP 183 503 and EP 154 316. The conjugation of the polypeptide and the activated polymer molecules is conducted by use of any conventional method, e.g. as described in the following references (which also describe suitable methods for activation of polymer molecules): R. F. Taylor, (1991), “Protein immobilisation. Fundamental and applications”, Marcel Dekker, N.Y.; S. S. Wong, (1992), “Chemistry of Protein Conjugation and Crosslinking”, CRC Press, Florida, USA; G. T. Hermanson et al., (1993), “Immobilized Affinity Ligand Techniques”, Academic Press, N.Y.). The skilled person will be aware that the activation method and/or conjugation chemistry to be used depends on the attachment group(s) of the polypeptide (examples of which are given further above), as well as the functional groups of the polymer (e.g. being amine, hydroxyl, carboxyl, aldehyde, sulfydryl, succinimidyl, maleimide, vinysulfone or haloacetate). The PEGylation may be directed towards conjugation to all available attachment groups on the polypeptide (i.e. such attachment groups that are exposed at the surface of the polypeptide) or may be directed towards one or more specific attachment groups, e.g. the N-terminal amino group (U.S. Pat. No. 5,985,265). Furthermore, the conjugation may be achieved in one step or in a stepwise manner (e.g. as described in WO 30 99/55377).

It will be understood that the PEGylation is designed so as to produce the optimal molecule with respect to the number of PEG molecules attached, the size and form of such molecules (e.g. whether they are linear or branched), and the attachment site(s) in the polypeptide peptide. The molecular weight of the polymer to be used may e.g. be chosen on the basis of the desired effect to be achieved. For instance, in the present invention a primary purpose is to achieve a conjugate having a high molecular weight (e.g. to reduce renal clearance). This may be achieved by conjugating few high Mw polymer molecules or a higher number of low Mw polymer molecules. When a high degree of epitope shielding is desirable this may be obtained by use of a sufficiently high number of low molecular weight polymer (e.g. with a molecular weight of about 5,000 Da) to effectively shield all or most epitopes of the polypeptide. For instance, 2-8, such as 3-6 such polymers may be used.

In connection with conjugation to only a single attachment group on the protein (as described in U.S. Pat. No. 5,985,265), it may be advantageous that the polymer molecule, which may be linear or branched, has a high molecular weight, e.g. about 20 kDa. Normally, the polymer conjugation is performed under conditions aimed at reacting all available polymer attachment groups with polymer molecules, in particular by using a molar excess of the macromolecular substance relative to the polypeptide. Typically, the molar ratio of activated polymer molecules to polypeptide is up to about 1000-1, in particular up to about 200-1, preferably up to about 100-1, such as up to about 10-1 or 5-1 in order to obtain optimal reaction. However, also equimolar ratios may be used.

It is also contemplated according to the invention to couple the polymer molecules to the polypeptide through a linker. Suitable linkers are well known to the skilled person. A preferred example is cyanuric chloride (Abuchowski et al., (1977), J. Biol. Chem., 252, 3578-3581; U.S. Pat. No. 4,179,337; Shafer et al., (1986), J. Polym. Sci. Polym. Chem. Ed., 24, 375-378).

Subsequent to the conjugation, residual activated polymer molecules are preferably blocked according to methods known in the art, e.g. by addition of primary amine to the reaction mixture, and the resulting inactivated polymer molecules are removed by a suitable method.

The general technology described in WO 99/55377 is also applicable for producing the conjugates of the present invention. Accordingly, in a further aspect the invention relates to a method for stepwise attachment of polyethylene glycol (PEG) moieties in series to a GH polypeptide of the invention, comprising the steps of:

reacting the polypeptide with a low molecular weight heterobifunctional or homobifunctional PEG moiety having the following formula: W—CH₂CH₂0(CH₂CH₂0)ₙCH₂CH₂—X, where W and X are groups that independently react with an amine, sulfhydryl, carboxyl or hydroxyl functional group to attach the low molecular weight PEG moiety to the polypeptide; and reacting the low molecular weight PEG moiety attached to the polypeptide with a monofunctional or bifunctional PEG moiety to attach the monofunctional or bifunctional PEG moiety to a free terminus of the low molecular weight PEG moiety and form a PEG-polypeptide conjugate. The “n” is an integer, which will depend on the weight of the low molecular weight PEG moiety. In one embodiment the monofunctional or bifunctional PEG moiety has the following formula: Y—CH₂CH₂0 (CH₂CH₂0), nCH₂CH₂-Z, wherein Y is reactive to a terminal group on the free terminus of the low molecular weight PEG moiety attached to the polypeptide and Z is—OCH3 or a group reactive with X to form a bifunctional conjugate. In a further embodiment the monofunctional or bifunctional PEG moiety is methoxy PEG, branched PEG, hydrolytically or enzymatically degradable PEG, pendant PEG, or dendrimer PEG. In a further embodiment W and X are independently selected from the group consisting of orthopyridyl disulfide, maleimides, vinylsulfones, iodoacetamides, hydrazides, aldehydes, succinimidyl esters, epoxides, amines, thiols, carboxyls, active esters, benzotriazole carbonates, p-nitrophenol carbonates, isocyanates, and biotin. In a further embodiment the low molecular weight PEG moiety has a molecular weight in a range of about 100 to 5,000 daltons, one example being OPSS-PEG-hydrazide. In a further embodiment the monofunctional or bifunctional PEG moiety has a molecular weight in a range of about 100 daltons to 200 kilodaltons. In a further embodiment the low molecular weight PEG moiety and/or the monofunctional or bifunctional PEG moiety is a copolymer of polyethylene glycol, such copolymer of polyethylene glycol is typically, selected from the group consisting of polyethylene glycol/polypropylene glycol copolymers and polyethylene glycol/poly (lactic/glycolic acid) copolymers. In a further embodiment the method further comprises a step of purifying the PEG-polypeptide conjugate following the stepwise attachment of two PEG moieties in series to the polypeptide. The term “OPSS-PEG-hydrazide in combination with mPEG-ALD” as used above and throughout this description is intended to means that the stepwise technologi disclosed in WO 99/55377 may be used. The disclosure of WO 99/55377 is incorporated herein by reference.

In order to avoid attachment of a polymer molecule in a functional site of the polypeptide GH (e.g. of the variant GH) of the invention, e.g. a receptor binding site thereof, it may be advantageous to shield such site during conjugation, also termed by blocking the functional site prior to conjugation (e.g. using the principle described in WO 94/13322). For instance, such site may be shielded by a monoclonal antibody. Thus, the functional site of the polypeptide may be blocked by a helper molecule capable of binding to the functional site of the polypeptide. Typically, the helper molecule is one, which specifically recognizes a functional site of the polypeptide, such as a receptor. Alternatively, the helper molecule may be an antibody, in particular a monoclonal antibody recognizing the polypeptide. In particular, the helper molecule may be a neutralizing monoclonal antibody. Preferably, the polypeptide is allowed to interact with the helper molecule before effecting conjugation. This ensures that the functional site of the polypeptide is shielded or protected and consequently unavailable for derivatization by the non-polypeptide moiety such, as a polymer. Following its elution from the helper molecule, the conjugate between the non-polypeptide moiety and the polypeptide can be recovered with at least a partially preserved functional site. The subsequent conjugation of the polypeptide having a blocked functional site to a polymer, a lipophilic compound, an organic derivatizing agent or any other compound is conducted in the normal way.

Irrespectively of the nature of the helper molecule to be used to shield the functional site of the polypeptide from conjugation, it is desirable that the helper molecule is free from or comprises only a few attachment groups for the non-polypeptide moiety of choice in part(s) of the molecule, where the conjugation to such groups will hamper the desorption of the conjugated polypeptide from the helper molecule. Hereby, selective conjugation to attachment groups present in non-shielded parts of the polypeptide can be obtained and it is possible to reuse the helper molecule for repeated cycles of conjugation. For instance, if the non-polypeptide moiety is a polymer molecule such as PEG, which has the epsilon amino group of a lysine or N-terminal amino acid residue as an attachment group, it is desirable that the helper molecule is substantially free from conjugatable epsilon amino groups, preferably free from any epsilon amino groups. Accordingly, in a preferred embodiment the helper molecule is a protein or peptide capable of binding to the functional site of the polypeptide, which protein or peptide is free from any conjugatable attachment groups for the non-polypeptide moiety of choice.

In a further embodiment the helper molecule is first covalently linked to a solid phase such as column packing materials, for instance Sephadex or agarose beads, or a surface, e.g. reaction vessel. Subsequently, the polypeptide is loaded onto the column material carrying the helper molecule and conjugation carried out according to methods known in the art. This procedure allows the polypeptide conjugate to be separated from the helper molecule by elution. The polypeptide conjugate is eluated by conventional techniques under physico-chemical conditions that do not lead to a substantive degradation of the polypeptide conjugate. The fluid phase containing the polypeptide conjugate is separated from the solid phase to which the helper molecule remains covalently linked. The separation can be achieved in other ways: For instance, the helper molecule may be derivatised with a second molecule (e.g. biotin) that can be recognized by a specific binder (e.g. streptavidin). The specific binder may be linked to a solid phase thereby allowing the separation of the polypeptide conjugate from the helper molecule-second molecule complex through passage over a second helper-solid phase column which will retain, upon subsequent elution, the helper molecule-second molecule complex, but not the polypeptide conjugate. The polypeptide conjugate may be released from the helper molecule in any appropriate fashion. De-protection may be achieved by providing conditions in which the helper molecule dissociates from the functional site of the polypeptide to which it is bound. For instance, a complex between an antibody to which a polymer is conjugated and an anti-idiotypic antibody can be dissociated by adjusting the pH to an acid or alkaline pH. Covalent in vitro coupling of a carbohydrate moiety to amino acid residues of polypeptide may be used to modify or increase the number or profile of carbohydrate substituents. Depending on the coupling mode used, the carbohydrate(s) may be attached to a) arginine and histidine (Lundblad and Noyes, Chemical Reagents for Protein Modification, CRC Press Inc. Boca Raton, Fla.), b) free carboxyl groups (e.g. of the C-terminal amino acid residue, asparagine or glutamine), c) free sulfhydryl groups such as that of cysteine, d) free hydroxyl groups such as those of serine, threonine, tyrosine or hydroxyproline, e) aromatic residues such as those of phenylalanine or tryptophan or f) the amide group of glutamine. These amino acid residues constitute examples of attachment groups for a carbohydrate moiety, which may be introduced in the GH polypeptide. Suitable methods of in vitro coupling are described in e.g. WO 87/05330 and in Aplin et al., CRC Crit. Rev. Biochem., pp. 259-306, 1981. The in vitro coupling of oligosaccharide moieties or PEG to protein- and peptide-bound Gln residues can also be carried out by transglutaminases (TGases). Transglutaminases catalyse the transfer of donor amine groups to protein- and peptide-bound Gln residues in a so-called cross-linking reaction. The donor-amine groups can be protein- or peptide-bound e.g. as the e-amino group in Lys-residues or can be part of a small or large organic molecule. An example of a small organic molecule functioning as amino donor in TGase-catalysed cross-linking is putrescine (1,4-diaminobutane). An example of a larger organic molecule functioning as amino donor in TGase-catalysed cross-linking is an amine-containing PEG (Sato et al., Biochemistry 35, 13072-13080).TGases, in general, are highly specific enzymes, and not every Gin residue exposed on the surface of a protein is accessible to TGase-catalysed cross-linking to amino-containing substances. On the contrary, only a few Gin residues function naturally as TGase substrates, but the exact parameters governing which Gin residues are good TGase substrates remain unknown. Thus, in order to render a protein susceptible to TGase-catalysed cross-linking reactions it is often a prerequisite at convenient positions to add stretches of amino acid sequence known to function well as TGase substrates. Several amino acid sequences are known to be or to contain excellent natural TGase substrates e.g. substance P, elafin, fibrinogen, fibronectin, α₂-plasmin inhibitor, α-caseins, and β-caseins.

Conjugation to an Organic Derivatizing Agent

Covalent modification of the GH polypeptide may be performed by reacting one or more attachment groups of the polypeptide with an organic derivatizing agent. Suitable derivatizing agents and methods are well known in the art. For example, cysteinyl residues most commonly are reacted with α-haloacetates (and corresponding amines), such as chloroacetic acid or chloroacetamide, to give carboxymethyl or carboxyamidomethyl derivatives. Cysteinyl residues also are derivatized by reaction with bromotrifluoroacetone, α-bromo-β-(4-imidozoyl)propionic acid, chloroacetyl phosphate, N-alkylmaleimides, 3-nitro-2-pyridyl disulfide, methyl 2-pyridyl disulfide, p-chloromercuribenzoate, 2-chloromercuri-4-nitrophenol, or chloro-7-nitrobenzo-2-oxa-1,3-diazole. Histidyl residues are derivatized by reaction with diethylpyrocarbonateat, pH 5.5-7.0, because this agent is relatively specific for the histidyl side chain. Para-bromophenacyl bromide is also useful. The reaction is preferably performed in 0.1 M sodium cacodylate at pH 6.0. Lysinyl and amino terminal residues are reacted with succinic or other carboxylic acid anhydrides. Derivatization with these agents has the effect of reversing the charge of the lysinyl residues. Other suitable reagents for derivatizing α-amino-containing residues include imidoesters such as methyl picolinimidate, pyridoxal phosphate, pyridoxal, chloroborohydride, trinitrobenzenesulfonic acid, O-methylisourea, 2,4-pentanedione and transaminase-catalyzed reaction with glyoxylate. Arginyl residues are modified by reaction with one or several conventional reagents, among them phenylglyoxal, 2,3-butanedione, 1,2-cyclohexanedione and ninhydrin. Derivatization of arginine residues requires that the reaction be performed in alkaline conditions because of the high pKa of the guanidine functional group.

Furthermore, these reagents may react with the groups of cysteine as well as the arginine guanidino group. Carboxyl side groups (aspartyl or glutamyl) are selectively modified by reaction with carbodiimides (R—N═C═N—R′), where R and R′ are different alkyl groups, such as 1-cyclohexyl-3-(2-morpholinyl-4-ethyl) carbodiimide or 1-ethyl-3-(4-azonia4,4-dimethylpentyl) carbodiimide. Furthermore, aspartyl and glutamyl residues are converted to asparaginyl and glutaminyl residues by reaction with ammonium ions.

Methods for Preparing a Polypeptide GH, Including a Variant GH

The polypeptide GH, such as the variant GH, used in accordance with the invention, optionally in glycosylated form, may be produced by any suitable method known in the art. Such methods include constructing a nucleotide sequence encoding the polypeptide and expressing the sequence in a suitable transformed or transfected host, e.g. as described by E. B. Jensen and S. Carlsen in Biotech and Bioeng. 36, 1-11 (1990). The polypeptide may be produced recombinantly (e.g. in E. coli) with an N-terminal extension such as Met-GH (e.g. Met-hGH), Met-Glu-Ala-GH (e.g. Met-Glu-Ala-hGH), Ala-Glu-GH (e.g. Ala-Glu-hGH) optionally followed by proteolytic cleavage to obtain hGH without the N-terminal extension before or after the attachment of the macromolular substance, e.g. before the N-terminally attachment of a macromolecular substance as described herein. In such embodiments, the GH polypeptide part of the conjugate of the invention does not comprise an N-terminal methionine, in particular for the medical uses as indicated herein.

However, polypeptides of the invention may be produced, albeit less efficiently, by chemical synthesis or a combination of chemical synthesis or a combination of chemical synthesis and recombinant DNA technology.

A nucleotide sequence encoding a variant GH of the invention may be synthesized on the basis of the amino acid sequence of the parent polypeptide, e.g. having the amino acid sequence shown in SEQ ID NO: 2, and then changing the nucleotide sequence so as to effect introduction (i.e. insertion or substitution) or removal (i.e. deletion or substitution) of the relevant amino acid residue(s). The nucleotide sequence may be conveniently modified by site-directed mutagenesis in accordance with conventional methods. Alternatively, the nucleotide sequence may be prepared by chemical synthesis, e.g. by using an oligonucleotide synthesizer, wherein oligonucleotides are designed based on the amino acid sequence of the desired polypeptide, and preferably selecting those codons that are favored in the host cell in which the recombinant polypeptide will be produced. For example, several small oligonucleotides coding for portions of the desired polypeptide may be synthesized and assembled by PCR, ligation or ligation chain reaction (LCR) (Barany, PNAS 88:189-193, 1991). The individual oligonucleotides typically contain 5′ or 3′ overhangs for complementary assembly. Once assembled (by synthesis, site-directed mutagenesis or another method), the nucleotide sequence encoding the polypeptide is inserted into a recombinant vector and operably linked to control sequences necessary for expression of the polypeptide in the desired transformed host cell. It should of course be understood that not all vectors and expression control sequences function equally well to express the nucleotide sequence encoding a polypeptide described herein. Neither will all hosts function equally well with the same expression system. However, one skilled in the art will be able to make a selection among these vectors, expression control sequences and hosts without undue experimentation. For example, in selecting a vector, the host must be considered because the vector must replicate in it or be able to integrate into the chromosome. The vector's copy number, the ability to control that copy number, and the expression of any other proteins encoded by the vector, such as antibiotic markers, should also be considered. In selecting an expression control sequence, a variety of factors should also be considered. These include, for example, the relative strength of the sequence, its controllability, and its compatibility with the nucleotide sequence encoding the polypeptide, particularly as regards potential secondary structures. Hosts should be selected by consideration of their compatibility with the chosen vector, the toxicity of the product coded for by the nucleotide sequence, their secretion characteristics, their ability to fold the polypeptide correctly, their fermentation or culture requirements, and ease of purification of the products encoded by the nucleotide sequence. The recombinant vector may be an autonomously replicating vector, i.e. a vector which exists as an extrachromosomal entity, the replication of which is independent of chromosomal replication, e.g. a plasmid. Alternatively, the vector is one which, when introduced into a host cell, is integrated into the host cell genome and replicated together with the chromosome(s) into which it has been integrated. The vector is preferably an expression vector in which the nucleotide sequence encoding the polypeptide of the invention is operably linked to additional segments required for transcription of the nucleotide sequence. The vector is typically derived from plasmid or viral DNA. A number of suitable expression vectors for expression in the host cells mentioned herein are commercially available or described in the literature. Useful expression vectors for eukaryotic hosts include, for example, vectors comprising expression control sequences from SV40, bovine papilloma virus, adenovirus and cytomegalovirus. Specific vectors are, e.g., pCDNA3.1(+)\Hyg (Invitrogen, Carlsbad, Calif., USA) and pCI-neo (Stratagene, La Jolla, Calif., USA). Useful expression vectors for yeast cells include the 2 μ plasmid and derivatives thereof, the POT1 vector (U.S. Pat. No. 4,931,373), the pJSO37 vector described in Okkels, Ann. New York Acad. Sci. 782, 202-207, 1996, and pPICZ A, B or C (Invitrogen). Useful vectors for insect cells include pVL941, pBG311 (Cate et al., “Isolation of the Bovine and Human Genes for Mullerian Inhibiting Substance and Expression of the Human Gene in Animal Cells”, Cell, 45, pp. 685-98, 1986), pBluebac 4.5 and pMelbac (both available from Invitrogen). Useful expression vectors for bacterial hosts include known bacterial plasmids, such as plasmids from E. coli, including pBR322, pET3a and pET12a (both from Novagen Inc., Wis., USA), wider host range plasmids, such as RP4, phage DNAs, e.g. the numerous derivatives of phage lambda, e.g. NM989, and other DNA phages, such as M13 and filamentous single stranded DNA phages.

Other vectors for use in this invention include those that allow the nucleotide sequence encoding the polypeptide to be amplified in copy number. Such amplifiable vectors are well known in the art. They include, for example, vectors able to be amplified by DHFR amplification (see, e.g., Kaufman, U.S. Pat. No. 4,470,461, Kaufman and Sharp, “Construction Of A Modular Dihydrafolate Reductase cDNA Gene: Analysis Of Signals Utilized For Efficient Expression”, Mol. Cell. Biol., 2, pp. 1304-19 (1982)) and glutamine synthetase (“GS”) amplification (see e.g. U.S. Pat. No. 5,122,464 and EP 338,841).

The recombinant vector may further comprise a DNA sequence enabling the vector to replicate in the host cell in question. An example of such a sequence (when the host cell is a mammalian cell) is the SV40 origin of replication. When the host cell is a yeast cell, suitable sequences enabling the vector to replicate are the yeast plasmid 2 μ replication genes REP 1-3 and origin of replication.

The vector may also comprise a selectable marker, e.g. a gene whose product complements a defect in the host cell, such as the gene coding for dihydrofolate reductase (DHFR) or the Schizosaccharomyces pombe TPI gene (described by P. R. Russell, Gene 40, 1985, pp. 125-130), or one which confers resistance to a drug, e.g. ampicillin, kanamycin, tetracyclin, chloramphenicol, neomycin, hygromycin or methotrexate. For Saccharomyces cerevisiae, selectable markers include ura3 and leu2. For filamentous fungi, selectable markers include amdS, pyrG, arcB, niaD and sC.

The term “control sequences” is defined herein to include all components that are necessary or advantageous for the expression of the polypeptide of the invention. Each control sequence may be native or foreign to the nucleic acid sequence encoding the polypeptide. Such control sequences include, but are not limited to, a leader sequence, polyadenylation sequence, propeptide sequence, promoter, enhancer or upstream activating sequence, signal peptide sequence, and transcription terminator. At a minimum, the control sequences include a promoter. A wide variety of expression control sequences may be used in the present invention. Such useful expression control sequences include the expression control sequences associated with structural genes of the foregoing expression vectors as well as any sequence known to control the expression of genes of prokaryotic or eukaryotic cells or their viruses, and various combinations thereof. Examples of suitable control sequences for directing transcription in mammalian cells include the early and late promoters of SV40 and adenovirus, e.g. the adenovirus 2 major late promoter, the MT-1 (metallothionein gene) promoter, the human cytomegalovirus immediate-early gene promoter (CMV), the human elongation factor 1α (EF-1α) promoter, the Drosophila minimal heat shock protein 70 promoter, the Rous Sarcoma Virus (RSV) promoter, the human ubiquitin C (UbC) promoter, the human growth hormone terminator, SV40 or adenovirus Elb region polyadenylation signals and the Kozak consensus sequence (Kozak, M. J Mol Biol 1987 Aug 20;196(4):947-50). In order to improve expression in mammalian cells a synthetic intron may be inserted in the 5′ untranslated region of the nucleotide sequence encoding the polypeptide. An example of a synthetic intron is the synthetic intron from the plasmid pCI-Neo (available from Promega Corporation, WI, USA). Examples of suitable control sequences for directing transcription in insect cells include the polyhedrin promoter, the P10 promoter, the Autographa californica polyhedrosis virus basic protein promoter, the baculovirus immediate early gene 1 promoter, the baculovirus 39K delayed-early gene promoter, and the SV40 polyadenylation sequence. Examples of suitable control sequences for use in yeast host cells include the promoters of the yeast α-mating system, the yeast triose phosphate isomerase (TPI) promoter, promoters from yeast glycolytic genes or alcohol dehydrogenase genes, the ADH2-4c promoter, and the inducible GAL promoter. Examples of suitable control sequences for use in filamentous fungal host cells include the ADH3 promoter and terminator, a promoter derived from the genes encoding Aspergillus oryzae TAKA amylase triose phosphate isomerase or alkaline protease, an A. niger α-amylase, A. niger or A. nidulans glucoamylase, A. nidulans acetamidase, Rhizomucor miehei aspartic proteinase or lipase, the TPI1 terminator and the ADH3 terminator. Examples of suitable control sequences for use in bacterial host cells include promoters of the lac system, the trp system, the TAC or TRC system, and the major promoter regions of phage lambda.

The presence or absence of a signal peptide will e.g. depend on the expression host cell used for the production of the polypeptide to be expressed (whether it is an intracellular or extracellular polypeptide) and whether it is desirable to obtain secretion. For use in filamentous fungi, the signal peptide may conveniently be derived from a gene encoding an Aspergillus sp. amylase or glucoamylase, a gene encoding a Rhizomucor miehei lipase or protease or a Humicola lanuginosa lipase. The signal peptide is preferably derived from a gene encoding A. oryzae TAKA amylase, A. niger neutral α-amylase, A. niger acid-stable amylase, or A. niger glucoamylase. For use in insect cells, the signal peptide may conveniently be derived from an insect gene (cf. WO 90/05783), such as the Lepidopteran manduca sexta adipokinetic hormone precursor, (cf. U.S. Pat. No. 5,023,328), the honeybee melittin (Invitrogen), ecdysteroid UDPglucosyltransferase (egt) (Murphy et al., Protein Expression and Purification 4, 349-357 (1993) or human pancreatic lipase (hpl) (Methods in Enzymology 284, pp. 262-272, 1997). A preferred signal peptide for use in mammalian cells is that of GH or the murine Ig kappa light chain signal peptide (Coloma, M (1992) J. Imm. Methods 152:89-104). For use in yeast cells, suitable signal peptides have been found to be the α-factor signal peptide from S. cereviciae (cf. U.S. Pat. No. 4,870,008), a modified carboxypeptidase signal peptide (cf. L. A. Valls et al., Cell 48, 1987, pp. 887-897), the yeast BAR1 signal peptide (cf. WO 87/02670), the yeast aspartic protease 3 (YAP3) signal peptide (cf. M. Egel-Mitani et al., Yeast 6, 1990, pp. 127-137), and the synthetic leader sequence TA57 (WO98/32867). For use in E. coli cells a suitable signal peptide has been found to be the signal peptide ompA (EP 581 821).

The nucleotide sequence of the invention encoding a polypeptide GH, in particular a variant GH, of the invention, whether prepared by site-directed mutagenesis, synthesis, PCR or other methods, may or may not also include a nucleotide sequence that encodes a signal peptide. The signal peptide is present when the polypeptide is to be secreted from the cells in which it is expressed. Such a signal peptide, if present, should be one recognized by the cell chosen for expression of the polypeptide. The signal peptide may be homologous (e.g. be that normally associated GH) or heterologous (i.e. originating from another source than human) to the polypeptide or may be homologous or heterologous to the host cell, i.e. be a signal peptide normally expressed from the host cell or one which is not normally expressed from the host cell. Accordingly, the signal peptide may be prokaryotic, e.g. derived from a bacterium such as E. coli, or eukaryotic, e.g. derived from a mammalian, insect or yeast cell.

Any suitable host may be used to produce the polypeptide GH of the invention (in particular the variant GH), including bacteria, fungi (including yeasts), plants, insects, mammals or other animals, or an appropriate animal cell line or another cell line. Examples of bacterial host cells include gram-positive bacteria such as strains of Bacillus, e.g. B. brevis or B. subtilis, Pseudomonas or Streptomyces, or gram-negative bacteria such as strains of E. coli. The introduction of a vector into a bacterial host cell may, for instance, be effected by protoplast transformation (see e.g. Chang and Cohen, 1979, Molecular General Genetics 168: 111-115), using competent cells (see e.g. Young and Spizizin, 1961, Journal of Bacteriology 81: 823-829, or Dubnau and Davidoff-Abelson, 1971, Journal of Molecular Biology 56:209-221), electroporation (see e.g. Shigekawa and Dower, 1988, Biotechniques 6: 742-751), or conjugation (see e.g. Koehler and Thorne, 1987, Journal of Bacteriology 169: 5771-5278).

Examples of suitable filamentous fungal host cells include strains of Aspergillus, e.g. A. oryzae, A. niger or A. nidulans, Fusarium or Trichoderma. Fungal cells may be transformed by a process involving protoplast formation, transformation of the protoplasts, and regeneration of the cell wall in a manner known per se. Suitable procedures for transformation of Aspergillus host cells are described in EP 238 023 and U.S. Pat. No. 5,679,543. Suitable methods for transforming Fusarium species are described by Malardier et al., 1989, Gene 78: 147-156 and WO 96/00787. Examples of suitable yeast host cells include strains of Saccharomyces, e.g. S. cerevisiae, Schizosaccharomyces, Klyveromyces, Pichia, such as P. pastoris or P. methanolica, Hansenula, such as H. polymorpha, or Yarrowia. Yeast may be transformed using the procedures described by Becker and Guarente, In Abelson, J. N. and Simon, M. I., editors, Guide to Yeast Genetics and Molecular Biology, Methods in Enzymology, Volume 194, pp. 182-187, Academic Press, Inc., New York; Ito et al., 1983, Journal of Bacteriology 153: 163; Hinnen et al., 1978, Proceedings of the National Academy of Sciences USA 75: 1920: and as disclosed by Clontech Laboratories, Inc., Palo Alto, Calif., USA (in the product protocol for the Yeastmaker™ Yeast Transformation System Kit). Examples of suitable insect host cells include a Lepidoptora cell line, such as Spodoptera frugiperda (Sf9 or Sf21) or Trichoplusioa ni cells (High Five) (U.S. Pat. No. 5,077,214). Transformation of insect cells and production of heterologous polypeptides therein may be performed as described by Invitrogen. Examples of suitable mammalian host cells include Chinese hamster ovary (CHO) cell lines, (e.g. CHO-K1; ATCC CCL-61), Green Monkey cell lines (COS) (e.g. COS 1 (ATCC CRL-1650), COS 7 (ATCC CRL-1651)); mouse cells (e.g. NS/O), Baby Hamster Kidney (BHK) cell lines (e.g. ATCC CRL-1632 or ATCC CCL-10), and human cells (e.g. HEK 293 (ATCC CRL-1573)), as well as plant cells in tissue culture. Also, the mammalian cell, such as a CHO cell, may be modified to express sialyltransferase, e.g. 1,6-sialyltransferase, e.g. as described in U.S. Pat. No. 5,047,335, in order to provide improved glycosylation of the GH polypeptide. Additional suitable cell lines are known in the art and available from public depositories such as the American Type Culture Collection, Rockville, Md., USA. Methods for introducing exogeneous DNA into mammalian host cells include calcium phosphate-mediated transfection, electroporation, DEAE-dextran mediated transfection, liposome-mediated transfection, viral vectors and the transfection method described by Life Technologies Ltd, Paisley, UK using Lipofectamin 2000. These methods are well known in the art and e.g. described by Ausbel et al. (eds.), 1996, Current Protocols in Molecular Biology, John Wiley & Sons, New York, USA. The cultivation of mammalian cells is conducted according to established methods, e.g. as disclosed in: Animal Cell Biotechnology, Methods and Protocols, Edited by Nigel Jenkins, 1999, Human Press Inc., Totowa, N.J., USA, and Harrison M A and Rae I F, General Techniques of Cell Culture, Cambridge University Press, 1997.

In the production methods of the present invention, the cells are cultivated in a nutrient medium suitable for production of the polypeptide using methods known in the art. For example, the cell may be cultivated by shake flask cultivation, small-scale or large-scale fermentation (including continuous, batch, fed-batch, or solid state fermentations) in laboratory or industrial fermentors performed in a suitable medium and under conditions allowing the polypeptide to be expressed and/or isolated. The cultivation takes place in a suitable nutrient medium comprising carbon and nitrogen sources and inorganic salts, using procedures known in the art. Suitable media are available from commercial suppliers or may be prepared according to published compositions (e.g. in catalogues of the American Type Culture Collection). If the polypeptide is secreted into the nutrient medium, the polypeptide can be recovered directly from the medium. If the polypeptide is not secreted, it can be recovered from cell lysates.

The resulting polypeptide may be recovered by methods known in the art. For example, the polypeptide may be recovered from the nutrient medium by conventional procedures including, but not limited to, centrifugation, filtration, extraction, spray drying, evaporation or precipitation. The polypeptides may be purified by a variety of procedures known in the art including, but not limited to, chromatography (e.g. ion exchange, affinity, hydrophobic, chromatofocusing, and size exclusion), electrophoretic procedures (e.g. preparative isoelectric focusing), differential solubility (e.g. ammonium sulfate precipitation), SDS-PAGE, or extraction (see e.g. Protein Purification, J.-C. Janson and Lars Ryden, editors, VCH Publishers, New York, 1989). Specific methods for purifying cytokine polypeptides are described in Human Cytokines, Handbook of Basic and Clinical Research, Volume II, Blackwell Science, Eds. Aggarwal and Gutterman, 1996, pp. 1942.

Homogeneous Preparation of a Conjugate of the Invention

Preferably, conjugates of the invention are provided in the form of a substantially homogeneous preparation. In the present context a “substantially homogeneous preparation” is a preparation, typically in a suitable buffer, containing more than 50%, such as more than 75% and preferably more than 85%, or more than 90% identical conjugates, i.e. having the same degree and nature of conjugation. The substantially homogeneous preparation is conveniently obtained by ensuring that the polypeptide GH of the invention, in particular the variant GH, contains the necessary number of attachment groups located at the surface of the molecule in such a way that all attachment groups can be conjugated to the macromolecular substance of choice when the conjugation is performed in the presence of a molar excess of the macromolecular substance relative to the polypeptide. Preferably, the macromolecular substance to be used in this aspect of the invention is a polymer molecule.

Pharmaceutical Use and Formulations

In a further aspect, the present invention relates to a pharmaceutical composition comprising the GH molecule, i.e. in particular the GH conjugate of the invention. The invention also relates to the GH molecule, i.e. in particular the GH conjugate or the pharmaceutical composition of the invention for use as a medicament. Accordingly, in one aspect the GH polypeptide, the GH conjugate or the pharmaceutical composition according to the invention is used for the manufacture of a medicament for treatment (i.e. including prevention as the case may be) of diseases, in particular for treatment of GH deficiency. Such diseases, i.e. including disorders, may involve inadequate growth caused by GH deficiency and/or GH insufficiency (e.g. GHD/GHI children). Also, a conjugate of the invention may be used in the treatment of Turner's syndrome, GH deficiency in adults (i.e. GHDA), Achondroplasia, Chronic Renal Insufficiency or Failure, including renal failure in children, AIDS waisting and treatment of cachexia in AIDS patients and cachexia associated with other diseases. The conjugate of the invention may also be used for the manufacture of a medicament for appetite suppression, e.g. in an individual on a low fat diet, optionally the medicament further comprises an antidiabetic agent or another appetite suppressing or satiety-inducing agent. Further, the conjugate of the invention may be used for the manufacture of a medicament for promoting bone formation in a mammal, in particular a human being, simultaneous with callus distraction. In further aspects, the conjugate of the invention may be used for the manufacture of a medicament for enhancing the healing of bone fractures in a mammal subjected to distraction osteogenesis, preferably wherein the conjugate of the invention is administered simultaneous with the distraction procedure. In further aspect, the conjugate of the invention is an antagonist of hGH and may be used for the manufacture of a medicament for treatment of diseases which involves excess production of GH, e.g. cancer or inflammation conditions. In another aspect, the polypeptide, in particular the conjugate, of the invention is used in a method for treating a mammal having diseases as described above, which method comprises administering to a mammal in need thereof such polypeptide, conjugate or pharmaceutical composition.

Therapeutic formulations of the GH polypeptide of the invention (including the pharmaceutical composition of the invention) are preferably administered in a composition that includes one or more pharmaceutically acceptable carriers or excipients. Such pharmaceutical compositions may be prepared in a manner known per se in the art to result in a polypeptide pharmaceutical that is sufficiently storage-stable and is suitable for administration to humans or animals. “Pharmaceutically acceptable” in the present context means a carrier or excipient that at the dosages and concentrations employed does not cause any untoward effects in the patients to whom it is administered. Such pharmaceutically acceptable carriers and excipients are well known in the art (see Remington's Pharmaceutical Sciences, 18th edition, A. R. Gennaro, Ed., Mack Publishing Company [1990]; Pharmaceutical Formulation Development of Peptides and Proteins, S. Frokjaer and L. Hovgaard, Eds., Taylor & Francis [2000]; and Handbook of Pharmaceutical Excipients, 3rd edition, A. Kibbe, Ed., Pharmaceutical Press [2000]). The formulation of the polypeptide of the invention may, e.g., be as described in WO9611702, WO9611703, WO9611704, WO9639173, WO 9702833, WO 9746252, WO 9703692 (e.g. a pharmaceutical composition comprising a GH polypeptide of the invention pre-treated with zinc and optionally lysine and calcium ions) or WO 9739768.

Drug Form

The polypeptide of the invention can be used “as is” and/or in a salt form thereof. Suitable salts include, but are not limited to, salts with alkali metals or alkaline earth metals, such as sodium, potassium, calcium and magnesium, as well as e.g. zinc salts. These salts or complexes may by present as a crystalline and/or amorphous structure.

Dose

The polypeptides and conjugates of the invention will be administered to patients in a pharmaceutically effective dose. By “pharmaceutically effective dose” herein is meant a dose that is sufficient to produce the desired effects in relation to the condition for which it is administered. The exact dose will depend on the disorder to be treated, and will be ascertainable by one skilled in the art using known techniques. In one embodiment, for example, the dose of the conjugate of the invention is corresponding to a dose in the range from about 0.001 to about 2.0 mg polypeptide per kg body weight, or about 0.01 to about 1.0 mg polypeptide per kg body weight, e.g. as a daily dosage, or preferably as a dosage administered less than daily e.g. 1-5 times a week, e.g. 1-3 or 1-2 times a week, e.g. 2-3 times a week or once a week, or at most or at least about every 5, 10, 15, 20, 25 or 30 days. The dose may be administered as a single dose or it may be administered in repeated doses during the day. For example treatment of diseases or conditions such as those listed above.

Mix of Drugs

The pharmaceutical composition of the invention may be administered alone or in conjunction with other therapeutic agents. These agents may be incorporated as part of the same pharmaceutical composition or may be administered separately from the polypeptide or conjugate of the invention, either concurrently or in accordance with another treatment schedule. In addition, the polypeptide, conjugate or pharmaceutical composition of the invention may be used as an adjuvant to other therapies.

Patients

A “patient” for the purposes of the present invention includes both humans and other mammals. Thus the methods are applicable to both human therapy and veterinary applications.

Types of Composition and Administration Route

The pharmaceutical composition of the polypeptide of the invention may be formulated in a variety of forms, e.g. as a liquid, gel, lyophilized, or as a compressed solid. The preferred form will depend upon the particular indication being treated and will be readily able to be determined by one skilled in the art.

The administration of the formulations of the present invention can be performed in a variety of ways, including, but not limited to, orally, subcutaneously, intravenously, intracerebrally, intranasally, transdermally, intraperitoneally, intramuscularly, intrapulmonary, or in any other acceptable manner. The formulations can be administered continuously by infusion, although bolus injection is acceptable, using techniques well known in the art, such as pumps or implantation. In some instances the formulations may be directly applied as a solution or spray.

Parenterals

An example of a pharmaceutical composition is a solution designed for parenteral administration. Although in many cases pharmaceutical solution formulations are provided in liquid form, appropriate for immediate use, such parenteral formulations may also be provided in frozen or in lyophilized form. In the former case, the composition must be thawed prior to use. The latter form is often used to enhance the stability of the active compound contained in the composition under a wider variety of storage conditions, as it is recognized by those skilled in the art that lyophilized preparations are generally more stable than their liquid counterparts. Such lyophilized preparations are reconstituted prior to use by the addition of one or more suitable pharmaceutically acceptable diluents such as sterile water for injection or sterile physiological saline solution.

In case of parenterals, they are preferably prepared for storage as lyophilized formulations or aqueous solutions by mixing, as appropriate, the polypeptide having the desired degree of purity with one or more pharmaceutically acceptable carriers, excipients or stabilizers typically employed in the art (all of which are termed “excipients”), for example buffering agents, stabilizing agents, preservatives, isotonifiers, non-ionic detergents, antioxidants and/or other miscellaneous additives.

Buffering agents help to maintain the pH in the range which approximates physiological conditions. They are typically present at a concentration ranging from about 2 mM to about 50 mM. Suitable buffering agents for use with the present invention include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyuconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additional possibilities are phosphate buffers, histidine buffers and trimethylamine salts such as Tris. Preservatives may be added to retard microbial growth, and are typically added in amounts of e.g. about 0.1%-2% (w/v). Suitable preservatives for use with the present invention include phenol, benzyl alcohol, meta-cresol, methyl paraben, propyl paraben, octadecyldimethylbenzyl ammonium chloride, benzalkonium halides (e.g. benzalkonium chloride, bromide or iodide), hexamethonium chloride, alkyl parabens such as methyl or propyl paraben, catechol, resorcinol, cyclohexanol and 3-pentanol. Isotonicifiers may be added to ensure isotonicity of liquid compositions and include polyhydric sugar alcohols, preferably trihydric or higher sugar alcohols, such as glycerin, erythritol, arabitol, xylitol, sorbitol and mannitol. Polyhydric alcohols can be present in an amount between 0.1% and 25% by weight, typically 1% to 5%, taking into account the relative amounts of the other ingredients.

Stabilizers may also be present and refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, omithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur-containing reducing agents, such as urea, glutathione, thioctic acid, sodium thioglycolate, thioglycerol, α-monothioglycerol and sodium thiosulfate; low molecular weight polypeptides (i.e. <10 residues); proteins such as human serum albumin, bovine serum albumin, gelatin or immunoglobulins; hydrophilic polymers such as polyvinylpyrrolidone; monosaccharides such as xylose, mannose, fructose and glucose; disaccharides such as lactose, maltose and sucrose; trisaccharides such as raffinose, and polysaccharides such as dextran. Stabilizers are typically present in the range of from 0.1 to 10,000 parts by weight based on the active protein weight.

Non-ionic surfactants or detergents (also known as “wetting agents”) may be present to help solubilize the therapeutic agent as well as to protect the therapeutic polypeptide against agitation-induced aggregation, which also permits the formulation to be exposed to shear surface stress without causing denaturation of the polypeptide. Suitable non-ionic surfactants include polysorbates (20, 80, etc.), polyoxamers (184, 188 etc.), Pluronic® polyols, poly-oxyethylene sorbitan monoethers (Tween®-20, Tween®-80, etc.).

Additional miscellaneous excipients include bulking agents or fillers (e.g. starch), chelating agents (e.g. EDTA), antioxidants (e.g., ascorbic acid, methionine, vitamin E) and cosolvents.

The active ingredient may also be entrapped in microcapsules prepared, for example, by coascervation techniques or by interfacial polymerization, for example hydroxymethyl-cellulose, gelatin or poly-(methylmethacylate) microcapsules, in colloidal drug delivery systems (for example liposomes, albumin microspheres, microemulsions, nano-particles and nanocapsules) or in macroemulsions. Such techniques are disclosed in Remington's Pharmaceutical Sciences, supra.

Parenteral formulations to be used for in vivo administration must be sterile. This is readily accomplished, for example, by filtration through sterile filtration membranes.

Sustained Release Preparations

Suitable examples of sustained-release preparations include semi-permeable matrices of solid hydrophobic polymers containing the polypeptide or conjugate, the matrices having a suitable form such as a film or microcapsules. Examples of sustained-release matrices include polyesters, hydrogels (for example, poly(2-hydroxyethyl-methacrylate) or poly(vinylalcohol)), polylactides, copolymers of L-glutamic acid and ethyl-L-glutamate, non-degradable ethylene-vinyl acetate, degradable lactic acid-glycolic acid copolymers such as the ProLease® technology or Lupron Depot® (injectable microspheres composed of lactic acid-glycolic acid copolymer and leuprolide acetate), and poly-D-(-)-3-hydroxybutyric acid. While polymers such as ethylene-vinyl acetate and lactic acid-glycolic acid enable release of molecules for long periods such as up to or over 100 days, certain hydrogels release proteins for shorter time periods. When encapsulated polypeptides remain in the body for a long time, they may denature or aggregate as a result of exposure to moisture at 37° C., resulting in a loss of biological activity and possible changes in immunogenicity. Rational strategies can be devised for stabilization depending on the mechanism involved. For example, if the aggregation mechanism is discovered to be intermolecular S—S bond formation through thio-disulfide interchange, stabilization may be achieved by modifying sulfhydryl residues, lyophilizing from acidic solutions, controlling moisture content, using appropriate additives, and developing specific polymer matrix compositions.

The invention is further described by the following non-limiting examples.

Methods

Methods Used to Determine the Amino Acids to be Modified

Accessible Surface Area (ASA)

The computer program Access (B. Lee and F. M. Richards, J. Mol.Biol. 55: 379-400 (1971)) version 2 (©1983 Yale University) is used to compute the accessible surface area (ASA) of the individual atoms in the structure. This method typically uses a probe-size of 1.4 Å and defines the Accessible Surface Area (ASA) as the area formed by the center of the probe. Prior to this calculation all water molecules and all hydrogen atoms should be removed from the coordinate set, as should other atoms not directly related to the protein.

Fractional ASA of Side Chain

The fractional ASA of the side chain atoms is computed by division of the sum of the ASA of the atoms in the side chain with a value representing the ASA of the side chain atoms of that residue type in an extended ALA-x-ALA tripeptide. See Hubbard, Campbell & Thornton (1991) J.Mol.Biol. 220, 507-530. For this example the CA atom is regarded as a part of the side chain of glycine residues but not for the remaining residues. The following values are used as standard 100% ASA for the side chain:

| | | |
|---|---|---|
| Ala | 69.23 | Å² |
| Arg | 200.35 | Å² |
| Asn | 106.25 | Å² |
| Asp | 102.06 | Å² |
| Cys | 96.69 | Å² |
| Gln | 140.58 | Å² |
| Glu | 134.61 | Å² |
| Gly | 32.28 | Å² |
| His | 147.00 | Å² |
| Ile | 137.91 | Å² |
| Leu | 140.76 | Å² |
| Lys | 162.50 | Å² |
| Met | 156.08 | Å² |
| Phe | 163.90 | Å² |
| Pro | 119.65 | Å² |
| Ser | 78.16 | Å² |
| Thr | 101.67 | Å² |
| Trp | 210.89 | Å² |
| Tyr | 176.61 | Å² |
| Val | 114.14 | Å² |

Residues not detected in the structure are typically defined as having 100% exposure as they are thought to reside in flexible regions.

Determining Distances Between Atoms

The distance between atoms is determined using molecular graphics software, e.g. InsightII v. 98.0, MSI Inc.

Methods Used to Determine the GH in Vitro and in Vivo Activity

In Vitro GH Activity Assay

In vitro GH activity may be determined by use of a cell line that proliferates in the presence of GH. For instance, such cell line is one expressing either the hGH receptor or a lactogenic receptor. For instance, the proliferation of the mouse pro-B cell line, Ba/F3-hGHR, expressing the human GH receptor (Wada et al., 1998, Mol. Endocrinol. 12, 146-156) or the rat Nb2 rat lymphoma cell line (Gout et al., 1980, Cancer Res. 40, 2433-2436) is useful for measuring GH activity. Furthermore, WO 99/03887 discloses the construction of useful cell lines for determining GH activity. See also Example 1 in WO 0042175 disclosing an in vitro assay for GH or Example XI of U.S. Pat. No. 6,004,931.

Measurement of the Functional in Vivo Half-Life

Functional in vivo half-life can be measured, e.g., by use of the “Analysis of Clearance in Rodents” protocol described by Clark et al., 1996, JBC, 271, 36, 21969-21977.

Antagonist GH Activity Assay

This may be measured as described in U.S. Pat. No. 6,004,931, e.g. in Example XII or example XIII.

Measurement of Receptor Binding

This may be measured as described in U.S. Pat. No. 6,004,931.

Determination of the Molecular Weight

The molecular weight may be determined by SDS-PAGE, gel filtration, matrix assisted laser desorption mass spectrometry or equilibrium centrifugation. A suitable SDS method is described by Laemmli, U.K., Nature Vol 227 (1970), p680-85.

The apparent molecular weight may also be estimated by gel permeation chromatography (GPC) by comparing the retention time of a component of interest to the retention time of various, preferably globular, protein standards (Protein purification methods, a practical approach (Harris & Angal, Eds.) IRL Press 1989, 293-306).

Methods for PEGylation of GH

PEGylation may be achieved by the method described by Clark et al., 1996, JBC, 271, 36, 21969-21977 or as described in WO 93/00109 (“Methods of hGH PEGylation”) or in WO 99/03887.

In Vivo Activity

WO 99/03887 discloses useful animal GH deficiency models of use for determining the in vivo activity of GH conjugates of the invention.

### EXAMPLES

### Example 1

The X-ray structure of hGH in complex with two copies of the extracellular part of the receptor bound to two different sites on the hGH molecule as reported by: Vos et.al. Science 255 (1992) 306-312, was used to determining positions into which introduce attachment sites for a macromolecular substance (such as PEG or an in vivo glycosylation site) or from which to remove such sites. The coordinates for this structure are available from the Protein Data Bank (PDB) (Bernstein et.al. J. Mol. Biol. (1977) 112 pp. 535) and electronically available via The Research Collaboratory for Structural Bioinformatics PDB at http://www.rcsb.org/pdb/ under accession code 3HHR. The structural part of the hGH molecule covers all of the mature sequence except the C-terminal F191 residue. Two disulphide bridges are present in the molecule connecting residues C53 with C165 and connecting C182 with C185. The binding sites of the two molecules of the extracellular part of human growth hormone receptor (hGHR) are thought to represent the binding mode for the activation. It is known that one hGHR molecule (labelled B) binds with high affinity to a site of hGH known as “site 1” and the other hGHR molecule (labelled C) binds with low affinity to a site of hGH known as “site 2”. Apparently hGH only binds to the other hGHR molecule after the first hGHR molecule is bound, possibly due to interactions with both the hGH and the already bound hGHR molecule (see Kossiakoff and Voss, Adv. in Prot. Chem. 52, 67-108, 1999 for a review).

Sequence Numbering:

The sequence numbering used in this example is according to the amino acid sequence of mature hGH shown in SEQ ID NO: 2.

Surface Exposure:

ASA calculations were performed on the hGH molecule alone (molecule A), on the complete complex consisting of the hGH molecule and two copies of the receptor molecule (molecules B and C), as well as well as of the two combinations of one molecule hGH and one molecule hGHR (molecules A and B and molecules A and C, respectively).

Performing fractional ASA calculations on the isolated hGH molecule (molecule A) resulted in the following residues having more than 25% of their side chain exposed to the surface; F1, P2, T3, I4, P5, L6, S7, R8, D11, N12, L15, R16, H18, R19, Q22, F25, D26, Q29, E30, E33, A34, Y35, P37, K38, E39, Q40, Y42, S43, L45, Q46, N47, P48, Q49, L52, E56, S57, P59, S62, N63, R64, E65, E66, Q68, Q69, K70, S71, E74, E88, Q91, F92, R94, S95, A98, N99, L101, Y103, G104, S106, D107, S108, N109, Y111, D112, K115, D116, E119, G120, Q122, T123, G126, R127, E129, D130, G131, P133, R134, T135, G136, Q137, K140, Q141, Y143, K145, D147, D154, A155, L156, K158, N159, G161, K168, D171, T175, R178, C182, R183, E186, G187, and G190. The following residues had more than 50% of their side chain exposed to the surface: F1, P2, T3, I4, P5, S7, R8, D11, N12, L15, H18, F25, Q29, E33, Y35, P37, K38, E39, Y42, S43, Q46, N47, P48, Q49, L52, S57, S62, N63, R64, E65, Q69, E88, Q91, R94, S95, N99, L101, Y103, S108, D112, K115, D116, E119, G120, Q122, G126, E129, G131, P133, R134, T135, G136, Y143, D147, D154, A155, K158, D171, T175, R178, E186, G187, and G190.

Performing fractional ASA calculations on the complex between hGH molecule and the two receptor (i.e. hGHR) molecules resulted in the following residues in hGH having more than 25% of their side chain exposed to the surface; F1, T3, P5, L6, S7, D11, Q22, D26, Q29, E30, E33, A34, Y35, P37, K38, E39, Q40, S43, Q46, N47, P48, Q49, L52, E56, S57, P59, N63, R64, E65, E66, Q69, K70, S71, E74, E88, Q91, F92, R94, S95, A98, N99, L101, Y103, G104, S106, D107, S108, N109, Y111, D112, K115, D116, E119, Q122, G126, R127, E129, D130, G131, P133, R134, T135, G136, Q137, K140, Q141, Y143, K145, D147, D154, A155, L156, K158, N159, G161, R183, E186, G187, and G190. The following residues had more than 50% of their side chain exposed to the surface: T3, P5, S7, Q29, E33, Y35, P37, K38, E39, S43, N47, P48, Q49, L52, S57, E65, Q69, E88, Q91, R94, S95, N99, L101, Y103, S108, D112, K115, Q122, G126, E129, G131, P133, R134, T135, G136, Y143, D147, D154, A155, K158, E186, G187, and G190.

Performing fractional ASA calculations on the complex between hGH molecule and the hGHR molecule B (the high affinity “site 1”) resulted in the following residues in hGH is having more than 25% of their side chain exposed to the surface; F1, P2, T3, I4, P5, L6, S7, R8, D11, N12, L15, R16, R19, Q22, D26, Q29, E30, E33, A34, Y35, P37, K38, E39, Q40, S43, Q46, N47, P48, Q49, L52, E56, S57, P59, N63, R64, E65, E66, Q69, K70, S71, E74, E88, Q91, F92, R94, S95, A98, N99, L101, Y103, G104, S106, D107, S108, N109, Y111, D112, K115, D116, E119, G120, Q122, T123, G126, R127, E129, D130, G131, P133, R134, T135, G136, Q137, K140, Q141, Y143, K145, D147, D154, A155, L156, K158, N159, G161, R183, E186, G187, G190. The following residues had more than 50% of their side chain exposed to the surface: F1, P2, T3, I4, P5, S7, R8, D11, N12, L15, Q29, E33, Y35, P37, K38, E39, S43, N47, P48, Q49, L52, S57, E65, Q69, E88, Q91, R94, S95, N99, L101, Y103, S108, D112, K115, D116, E119, G120, Q122, G126, E129, G131, P133, R134, T135, G136, Y143, D147, D154, A155, K158, E186, G187, G190.

Performing fractional ASA calculations on the complex between hGH molecule and the hGHR molecule C (the low affinity “site 2”) resulted in the following residues in hGH having more than 25% of their side chain exposed to the surface; F1, T3, P5, L6, S7, D11, H18, Q22, F25, D26, Q29, E30, E33, A34, Y35, P37, K38, E39, Q40, Y42, S43, L45, Q46, N47, P48, Q49, L52, E56, S57, P59, S62, N63, R64, E65, E66, Q68, Q69, K70, S71, E74, E88, Q91, F92, R94, S95, A98, N99, L101, Y103, G104, S106, D107, S108, N109, Y111, D112, K115, D116, E119, Q122, G126, R127, E129, D130, G131, P133, R134, T135, G136, Q137, K140, Q141, Y143, K145, D147, D154, A155, L156, K158, N159, G161, K168, D171, T175, R178, C182, R183, E186, G187, G190. The following residues had more than 50% of their side chain exposed to the surface: T3, P5, S7, H18, F25, Q29, E33, Y35, P37, K38, E39, Y42, S43, Q46, N47, P48, Q49, L52, S57, S62, N63, R64, E65, Q69, E88, Q91, R94, S95, N99, L101, Y103, S108, D112, K115, Q122, G126, E129, G131, P133, R134, T135, G136, Y143, D147, D154, A155, K158, D171, T175, R178, E186, G187, G190.

Receptor Binding Site:

Residues in hGH that have side chain atoms interacting with a receptor molecule can be defined as those residues having their side chain ASA changed in the ASA calculations of the complex as compared to the ASA calculations on the isolated hGH molecule. Those residues are: F1, P2, I4, P5, R8, L9, D11, N12, A13, M14, L15, R16, H18, R19, H21, Q22, F25, D26, Q29, K41, Y42, L45, Q46, P48, S51, E56, S62, N63, R64, E65, T67, Q68, Y103, D116, L117, E119, G120, T123, L124, R127, Y164, R167, K168, D171, K172, E174, T175, F176, R178, I179, C182, R183, E186, C189, G190. Expressed differently, these residues are believed to belong to the receptor binding site. The residues in this list still having more than 25% side chain ASA; F1, P5, D11, Q22, D26, Q29, Q46, P48, E56, N63, R64, E65, Y103, D116, E119, R127, R183, E186, G190, or even more than 50% side chain ASA; P5, Q29, P48, E65, Y103, E186, G190 are considered as being placed on the edge of the receptor binding sites.

The receptor binding site may be split in two, based on the interactions with each of the two hGHR molecules in the structure. Based on the same considerations as above the residues having changed side chain ASA between the isolated hGH and the calculation including only the B molecule (the high affinity “site 1”) are: M14, H18, H21, Q22, F25, D26, Q29, K41, Y42, L45, Q46, P48, S51, E56, S62, N63, R64, E65, T67, Q68, Y164, R167, K168, D171, K172, E174, T175, F176, R178, I179, C182, R183, E186, C189, G190. Expressed differently these residues are believed to belong the receptor binding “site 1”. Residues having changed side chain ASA between the isolated hGH and the calculation including only the C molecule (the low affinity “site 2”) are: F1, P2, I4, P5, R8, L9, D11, N12, A13, L15, R16, H18, R19, Q22, Y103, D116, L117, E119, G120, T123, L124, R127. Expressed differently these residues are believed to belong the receptor binding “site 2”. As can be seen from these lists residues H18 and Q22 have interactions with both receptor molecules.

Introduction/Removal of Lysine Residues

Removal of Lysine Residues

hGH contains 9 lysines. In one embodiment one or more of the lysine residues are removed, preferably by substitution to any other amino acid residue, but preferably arginine, in order to avoid attachment of a macromolecular substance to such residue(s). Accordingly, at least one lysine residue selected from the group consisting of K38, K41, K70, K115, K140, K145, K158, K168 and K172 are removed. Of particular interest is to remove one or more lysines of a receptor binding site, i.e. a lysine selected from the group consisiting of K41, K168 and K172.

Introduction of Lysine, Preferably by Substitution, to Introduce Attachment Group

Substitutions of surface exposed residues to lysine residues will introduce new potential attachment points for lysine reactive macromolecular substances. Of particular interest are residues that have their side chain exposed to the surface in the structure of the complex between hGH and the two receptor molecules. More preferably residues having more than 25% side chain ASA even more preferably residues having more than 50% side chain ASA. Preferably, one or more lysine residues are introduced by substitution of one or more of the amino acid residues identified in the below lists, e.g. 1, 2, 3, 4, 5 or more lysine residues.

The following non lysine residues have more than 25% side chain ASA and are targets for substitution to lysine: F1, T3, P5, L6, S7, D11, Q22, D26, Q29, E30, E33, A34, Y35, P37, E39, Q40, S43, Q46, N47, P48, Q49, L52, E56, S57, P59, N63, R64, E65, E66, Q69, S71, E74, E88, Q91, F92, R94, S95, A98, N99, L101, Y103, G104, S106, D107, S108, N109, Y111, D112, D116, E119, Q122, G126, R127, E129, D130, G131, P133, R134, T135, G136, Q137, Q141, Y143, D147, D154, A155, L156, N159, G161, R183, E186, G187, and G190. More preferably non lysine residues having more than 50% side chain ASA; T3, P5, S7, Q29, E33, Y35, P37, E39, S43, N47, P48, Q49, L52, S57, E65, Q69, E88, Q91, R94, S95, N99, L101, Y103, S108, D112, Q122, G126, E129, G131, P133, R134, T135, G136, Y143, D147, D154, A155, E186, G187, G190. From these lists it is more preferable to make the substitution at a position containing an arginine residue, i.e. R64, R94, R127, R134, R183, even more preferably R94 and/or R134.Furthermore, it is preferable not to make mutations at positions where the residue side chain is defined as being part of the receptor interface. This results in the following non-lysine residues having more than 25% side chain ASA being target for mutagenesis; T3, L6, S7, E30, E33, A34, Y35, P37, E39, Q40, S43, N47, Q49, L52, S57, P59, E66, Q69, S71, E74, E88, Q91, F92, R94, S95, A98, N99, L101, G104, S106, D107, S108, N109, Y111, D112, Q122, G126, E129, D130, G131, P133, R134, T135, G136, Q137, Q141, Y143, D147, D154, A155, L156, N159, G161, G187, T142. More preferably non lysine residues having more than 50% side chain ASA; T3, S7, E33, Y35, P37, K38, E39, S43, N47, Q49, L52, S57, Q69, E88, Q91, R94, S95, N99, L101, S108, D112, K115, Q122, G126, E129, G131, P133, R134, T135, G136, Y143, D147, D154, A155, K158, G187. From these lists it is more preferable to make the substitution at a position containing an arginine residue, i.e. R94 and/or R134.

Glycosylation Sites

Introduction of N-Glycosylation Sites

hGH does not contain any N-glycosylation sites. N-glycosylation sites can be introduced by mutations of one or two residues as described above, in particular under the sections “Definitions” and “Conjugate of the invention wherein the macromolecular substance is an oligosaccharide moiety”. Sites where the residue to be an “N” has more than 25% side chain ASA in the calculation on the complex between hGH and the two receptor molecules and “X” and “Z” is not P and none of the residues to be mutated is a Cys involved in a disulphide bridge are targets for mutagenesis to introduce the N-residue of a glycosylation site: T3, P5, L6, S7, D11, Q22, D26, Q29, E30, E33, Y35, P37, K38, E39, Q40, S43, Q46, P48, Q49, L52, S57, P59, N63, R64, E65, E66, Q69, K70, S71, E74, Q91, F92, R94, S95, A98, N99, L101, Y103, G104, S106, D107, S108, N109, Y111, D112, K115, D116, E119, Q122, G126, R127, E129, G131, P133, R134, T135, G136, Q137, K140, Q141, Y143, K145, D147, D154, A155, L156, K158, N159, G161, R183, and E186. This may be performed by corresponding mutations, i.e. selected from the group consisting of: T3N+P5S/T, P5N, P5N+S5T, L6N+R8S/T, S7N+L9S/T, D11N+A13S/T, Q22N+A24S/T, D26N+Y28S/T, Q29N+F31S/T, E30N+E32S/T, E33N+Y35S/T, Y35N+P37S/T, P37N+E39S/T, K38N+Q40S/T, E39N+K41S/T, Q40N+Y42S/T, S43N+L45S/T, Q46N+P48S/T, P48N+T50S, P48N, Q49N, Q49N+S51T, L52N+F54S/T, S57N+P59S/T, P59N+P61S/T, E65S/T, R64N+E66S/T, E65N+T67S, E65N, E66N+Q68S/T, Q69N, Q69N+S71T, K70N+N72S/T, S71N+L73S/T, E74N+L76S/T, Q91N+L93S/T, F92N+R94S/T, R94N+V96S/T, S95N+F97S/T, A98N, A98N+S100T, L101S/T, L101N+Y103S/T, Y103N+A105S/T, G104N, G104N+S106T, S106N, S106N+S108T, D107N+N109S/T, S108N+V110S/T, Y111S/T, Y111N+L113S/T, D112N+L114S/T, K115N+L117S/T, D116N+E118S/T, E119N+I121S/T, Q122N+L124S/T, G126N+L128S/T, R127N+E129S/T, E129N+G131S/T, G131N+P133S/T, P133N+T135S, P133N, R134N+G136S/T, T135N+Q137S/T, G136N+I138S/T, Q137N+F139S/T, K140N+T142S, K140N, Q141N+Y143S/T, Y143N+K145S/T, K145N+D147S/T, D147N+N149S/T, D154N+L156S/T, A155N+L157S/T, L156N+K158S/T, K158N+Y160S/T, G161S/T, G161N+L163S/T, R183N+V185S/T, E186N, and E186N+S188T. Even more preferred are positions where the residue to be an “N” has more than 50% side chain ASA; T3, P5, S7, Q29, E33, Y35, P37, K38, E39, S43, P48, Q49, L52, S57, E65, Q69, Q91, R94, S95, N99, L101, Y103, S108, D112, K115, Q122, G126, E129, G131, P133, R134, T135, G136, Y143, D147, D154, A155, K158, and E186. This may be performed by corresponding mutations, i.e. selected from the group consisting of: T3N+P5S/T, P5N, P5N+S5T, S7N+L9S/T, Q29N+F31S/T, E33N+Y35S/T, Y35N+P37S/T, P37N+E39S/T, K38N+Q40S/T, E39N+K41S/T, S43N+L45S/T, P48N+T50S, P48N, Q49N, Q49N+S51T, L52N+F54S/T, S57N+P59S/T, E65N+T67S, E65N, Q69N, Q69N+S71T, Q91N+L93S/T, F92N+R94S/T, R94N+V96S/T, S95N+F97S/T, L101S/T, L101N+Y103S/T, Y103N+A105S/T, S108N+V110S/T, D112N+L114S/T, K115N+L117S/T, Q122N+L124S/T, G126N+L128S/T, E129N+G131S/T, G131N+P133S/T, P133N+T135S, P133N, R134N+G136S/T, T135N+Q137S/T, G136N+I138S/T, Y143N+K145S/T, D147N+N149S/T, D154N+L156S/T, A155N+L157S/T, K158N+Y160S/T, E186N, and E186N+S188T.

From these lists it is more preferable to introduce N-glycosylation sites at positions already holding a N or an S/T in the “position 1” or “position 3”: P5, P48, Q49, N63, E65, Q69, A98, N99, G104, S106, N109, P133, K140, N159, and E186 having more than 25% side chain ASA more preferably P5, P48, Q49, E65, Q69, N99, P133, and E186 having more than 50% side chain ASA. This may be performed by corresponding mutations, i.e. selected from the group consisting of: P5N, P48N, Q49N, E65S/T, E65N, Q69N, A98N, L101S/T, G104N, S106N, Y111S/T, P133N, K140N, G161S/T, and E186N, more preferably selected from the group consisting of P5N, P48N, Q49N, E65N, Q69N, L101S/T, P133N, and E186N.

Accordingly, for any other position in the above lists, which do not comprise an S or a T residue in position +2 relative to the introduced N residue, it will be understood that an S or more preferably a T residue is to be introduced in such position (in addition to introduction of the N residue).

Furthermore, it may be preferable not to introduce an N-glycosylation site at positions where the residue to be an “N” has its side chain defined as being part of the receptor interface. This results in the following positions (having more than 25% of the side chain ASA in the calculation on the complex between hGH and the two receptor molecules and “X” and “Z” is not P and none of the residues to be mutated is a Cys involved in a disulphide bridge) are targets for mutagenesis to introduce a new potential glycosylation site: T3, L6, S7, E30, E33, Y35, P37, K38, E39, Q40, S43, Q49, L52, S57, P59, E66, Q69, K70, S71, E74, Q91, F92, R94, S95, A98, N99, L101, G104, S106, D107, S108, N109, Y111, D112, K115, Q122, G126, E129, G131, P133, R134, T135, G136, Q137, K140, Q141, Y143, K145, D147, D154, A155, L156, K158, N159, and/or G161. This may be performed by corresponding mutations, i.e. selected from the group consisting of: T3N+P5S/T, L6N+R8S/T, S7N+L9S/T, E30N+E32S/T, E33N+Y35S/T, Y35N+P37S/T, P37N+E39S/T, K38N+Q40S/T, E39N+K41S/T, Q40N+Y42S/T, S43N+L45S/T, Q49N, Q49N+S51T, L52N+F54S/T, S57N+P59S/T, P59N+P61S/T, E66N+Q68S/T, Q69N, Q69N+S71T, K70N+N72S/T, S71N+L73S/T, E74N+L76S/T, Q91N+L93S/T, F92N+R94S/T, R94N+V96S/T, S95N+F97S/T, A98N, A98N+S100T, L101S/T, L101N+Y103S/T, G104N, G104N+S106T, S106N, S106N+S108T, D107N+N109S/T, S108N+V110S/T, Y111S/T, Y111N+L113S/T, D112N+L114S/T, K115N+L117S/T, Q122N+L124S/T, G126N+L128S/T, E129N+G131S/T, G131N+P133S/T, P133N+T135S, P133N, R134N+G136S/T, T135N+Q137S/T, G136N+I138S/T, Q137N+F139S/T, K140N+T142S, K140N, Q141N+Y143S/T, Y143N+K145S/T, K145N+D147S/T, D147N+N149S/T, D154N+L156S/T, A155N+L157S/T, L156N+K158S/T, K158N+Y160S/T, G161S/T, and G161N+L163S/T.

Even more preferred are positions where the residue to be an “N” has more than 50% side chain ASA; T3, S7, E33, Y35, P37, K38, E39, S43, Q49, L52, S57, Q69, Q91, R94, S95, N99, L101, S108, D112, K115, Q122, G126, E129, G131, P133, R134, T135, G136, Y143, D147, D154, A155, and/or K158. This may be performed by corresponding mutations, i.e. selected from the group consisting of: T3N+P5S/T, S7N+L9S/T, E33N+Y35S/T, Y35N+P37S/T, P37N+E39S/T, K38N+Q40S/T, E39N+K41S/T, S43N+L45S/T, Q49N, Q49N+S51T, L52N+F54S/T, S57N+P59S/T, Q69N, Q69N+S71T, Q91N+L93S/T, R94N+V96S/T, S95N+F97S/T, L101S/T, L101N+Y103S/T, S108N+V110S/T, D112N+L114S/T, K115N+L117S/T, Q122N+L124S/T, G126N+L128S/T, E129N+G131S/T, G131N+P133S/T, P133N+T135S, P133N, R134N+G136S/T, T135N+Q137S/T, G136N+I138S/T, Y143N+K145S/T, D147N+N149S/T, D154N+L156S/T, A155N+L157S/T, K158N+Y160S/T.

From these lists it is more preferable to introduce N-glycosylation sites at positions already holding a N or an S/T in the “position 1” or “position 3”: Q49, Q69, A98, N99, G104, S106, N109, P133, K140, and/or N159 having more than 25% side chain ASA more preferably Q49, Q69, N99, and/or P133 having more than 50% side chain ASA. This may be performed by corresponding mutations, i.e. selected from the group consisting of: Q49N, Q69N, A98N, L101S/T, G104N, S106N, Y111S/T, P133N, K140N, G161S/T more preferably Q49N, Q69N, L101S/T, P133N.

1. A conjugate of a growth hormone polypeptide variant (variant GH) comprising at least one introduced non-cysteine amino acid residue, which residue comprises an attachment group for a (first) macromolecular substance, the residue having been introduced into a position of a parent growth hormone polypeptide (parent GH) that is equivalent to a surface exposed position of wildtype human growth hormone (hGH), the conjugate further comprising at least one (first) macromolecular substance reactive with the non-cysteine amino acid residue.

2.-13. (canceled)

14. A conjugate of a growth hormone polypeptide, wherein said polypeptide comprises at least one amino acid residue with an attachment group for a (first) macromolecular substance, which amino acid residue is located in a position that is equivalent to a surface exposed position in a helix of hGH, wherein the position is not located in a position equivalent to the first three or last three amino acid residues of said helix, the conjugate further comprising the macromolecular substance attached to said at least one amino acid residue.

15.-27. (canceled)

28. A conjugate of a variant GH comprising at least one introduced cysteine residue, which residue has been introduced in a position of a parent GH that is equivalent to a surface exposed position in a helix of hGH, preferably selected from the group consisting of A, B, C, and D, provided said position is not located in the first three or last three amino acid residues of said helix, the conjugate further comprising at least one (first) cysteine reactive macromolecular substance.

29.-34. (canceled)

35. A conjugate of a growth hormone polypeptide variant (variant GH) comprising at least one removed non-cysteine amino acid residue comprising an attachment group for a (first) macromolecular substance, the residue having been removed from a position of a parent growth hormone polypeptide (parent GH) that is equivalent to a surface exposed position of wildtype human growth hormone (hGH), the conjugate further comprising at least one (first) macromolecular substance attached to a non-cysteine amino acid residue present in said polypeptide, which macromolecular substance is reactive with the removed non-cysteine amino acid residue.

36.-43. (canceled)

44. A conjugate of a growth hormone polypeptide comprising at least one macromolecular substance attached to an attachment group of an amino acid residue in a position that is equivalent to a surface exposed position in the receptor binding site 2 of hGH, the conjugate further comprising at least one (first) macromolecular substance reactive with said amino acid residue.

45.-59. (canceled)

60. A conjugate of a variant GH comprising at least one introduced in vivo glycosylation site, the in vivo glycosylation site having been introduced in a position of a parent GH that is equivalent to a surface exposed position of hGH, the conjugate further comprising at least one oligosaccharide moiety.

61.-75. (canceled)

76. A nucleotide sequence encoding the variant GH claim 1.

77.-88. (canceled)

89. A nucleotide sequence encoding the growth hormone polypeptide of claim 14.

90. A nucleotide sequence encoding the variant GH of claim 28.

91. A nucleotide sequence encoding the growth hormone polypeptide variant of claim 35.

92. A nucleotide sequence encoding the growth hormone polypeptide of claim 44.

93. A nucleotide sequence encoding the variant GH of claim 60.


SEQUENCE LISTING (3 sequences)

SEQ ID NO: 1 - protein, 217 aa, Homo sapiens
MATGSRTSLLLAFGLLCLPWLQEGSAFPTIPLSRLFDNAMLRAHRLHQLAFDTYQEFEEAYIPKEQKYSFLQNPQTSLCFSESIPTPSNREETQQKSNLELLRISLLLIQSWLEPVQFLRSVFANSLVYGASDSNVYDLLKDLEEGIQTLMGRLEDGSPRTGQIFKQTYSKFDTNSHNDDALLKNYGLLYCFRKDMDKVETFLRIVQCRSVEGSCGF

SEQ ID NO: 2 - protein, 191 aa, Homo sapiens [sequence omitted]

SEQ ID NO: 3 - DNA, 654 bp, Homo sapiens
atggctacaggctcccggacgtccctgctcctggcttttggcctgctctgcctgccctggcttcaagagggcagtgccttcccaaccattcccttatccaggctttttgacaacgctatgctccgcgcccatcgtctgcaccagctggcctttgacacctaccaggagtttgaagaagcctatatcccaaaggaacagaagtattcattcctgcagaacccccagacctccctctgtttctcagagtctattccgacaccctccaacagggaggaaacacaacagaaatccaacctagagctgctccgcatctccctgctgctcatccagtcgtggctggagcccgtgcagttcctcaggagtgtcttcgccaacagcctggtgtacggcgcctctgacagcaacgtctatgacctcctaaaggacctagaggaaggcatccaaacgctgatggggaggctggaagatggcagcccccggactgggcagatcttcaagcagacctacagcaagttcgacacaaactcacacaacgatgacgcactactcaagaactacgggctgctctactgcttcaggaaggacatggacaaggtcgagacattcctgcgcatcgtgcagtgccgctctgtggagggcagctgtggcttctag